(19) United States
(12) Patent Application Publication
(10) Pub. No.: US 2026/0183372 A1
(43) Pub. Date: Jul. 2, 2026

(54) BACTERIAL LYSINS AND USES THEREOF

(71) Applicant: Huazhong Agricultural University, Wuhan, Hubei (CN)

(72) Inventors: Jinquan LI, Wuhan (CN); Geng ZOU, Wuhan (CN); Yue ZHANG, Wuhan (CN); Yang ZHOU, Wuhan (CN); Xingao HUANG, Wuhan (CN)

(21) Appl. No.: 19/260,213

(22) Filed: Jul. 3, 2025

Related U.S. Application Data
(63) Continuation of application No. PCT/CN2024/071450, filed on Jan. 9, 2024.

(30) Foreign Application Priority Data
Jan. 9, 2023 (CN) 202310037521.3

(51) Int. Cl. A61K 38/47; A61P 31/04; C12N 1/06; C12N 1/20; C12N 9/36; C12N 15/52; C12N 15/63; C12R 1/44; C12R 1/46

(52) U.S. Cl. CPC A61K 38/47; A61P 31/04; C12N 1/06; C12N 1/20; C12N 9/2462; C12N 15/52; C12N 15/63; C12Y 302/01017; C12R 2001/44; C12R 2001/46

(57) ABSTRACT

A general formula for lysins based on which the activity and functionality are further screened and 24 lysins are identified and further investigated, nine of which are characterized. These lysins exhibit potent bactericidal activity, capable of lysing multiple species of Staphylococcus, Streptococcus, and/or Enterococcus; demonstrate robust environmental resistance and stability, retaining activity under varying temperatures, NaCl concentrations, pH levels, and in immune serum; and effectively treat localized and systemic infections. These lysins and their variants are promising as antimicrobial agents for eradicating bacteria, preventing bacterial infections, or treating bacterial infections (caused by genera Staphylococcus, Streptococcus, and/or Enterococcus).

### CROSS-REFERENCE TO RELATED APPLICATIONS

The subject application is a continuation of PCT/CN2024/071450 filed on Jan. 9, 2024, which in turn claims priority on Chinese Patent Application No. CN 202310037521.3 filed on Jan. 9, 2023 in China. The contents and subject matters of the PCT international stage application and the Chinese priority application are incorporated herein by reference in the entirety.

### REFERENCE TO AN ELECTRONIC SEQUENCE LISTING

The contents of the electronic sequence listing (Name of the File: RevisedSequenceListing8028wh.xml; Size: 73,253 Bytes; and Date of Creation: Mar. 19, 2026) is herein incorporated by reference in its entirety.

### TECHNICAL FIELD

The present invention relates to the field of biological agents and bio-disinfectants, and specifically relates to bacterial lysins and their uses.

### BACKGROUND ART

Staphylococcus aureus is a Gram-positive pathogenic bacterium capable of causing diverse diseases ranging from skin and soft tissue infections to deep tissue abscesses, endocarditis, osteomyelitis, meningitis, and bacteremia. Studies indicate that in 2019, there are an estimated 4.95 million deaths associated with bacterial antimicrobial resistance (AMR), including 1.27 million deaths attributable to bacterial AMR. Among them, methicillin-resistant Staphylococcus aureus (MRSA) directly contributes to over 100,000 fatalities.

Streptococcus suis is a significant zoonotic pathogen. Outbreaks of human Streptococcus suis infections occurred in Jiangsu Province in 1998, Sichuan Province in 2005, and Guangxi Province in 2016. Consequently, there is an urgent need for novel antimicrobial agents with bactericidal activity against Staphylococcus aureus and Streptococcus suis.

Lysins, widely recognized as natural antimicrobial proteins, specifically target and kill pathogenic microorganisms without harming beneficial gut microbiota. Furthermore, no bacterial resistance to lysins has been reported to date.

The lysin LysRODI reported by Gutiérrez et al. exhibits a MIC₅₀ of 1.15 μM (63.03 μg/mL) and MIC₉₀ of 1.15 μM (63.03 μg/mL) against Staphylococcus aureus, with a minimum inhibitory concentration (MIC) of 0.57 μM (31.24 μg/mL) against Staphylococcus aureus ATCC 25923. LysRODI retains 50% enzymatic activity after 3 months of storage at 4° C. (Gutiérrez et al., 2021). ClyRODI-Lyso, a lysin sharing the same catalytic domain as LysRODI, demonstrates MIC₅₀ and MIC₉₀ values >14.54 μM (438.96 μg/mL) against Staphylococcus aureus (Gutiérrez et al., 2021). LysK, a lysin with 98.38% similarity to LysRODI, exhibits a MIC of 32.85±4.87 μg/mL against methicillin-resistant Staphylococcus aureus (MRSA) USA300 (Becker, Foster-Frey & Donovan, 2008), and lyses all tested Staphylococcus strains (10 Staphylococcus aureus, 1 Staphylococcus epidermidis, 1 Staphylococcus saprophyticus), but fails to lyse Streptococcus strains (Jun et al., 2011).

Additionally, the lysin CF-301 shows MIC₅₀ and MIC₉₀ values of 32 μg/mL against MRSA and 16 μg/mL (MIC₅₀) and 32 μg/mL (MIC₉₀) against methicillin-sensitive Staphylococcus aureus (MSSA), with an MIC of 64 μg/mL against ATCC43300 (Indiani et al., 2019; CN 104736172 A). ClyF, a lysin sharing the same binding domain as CF-301, exhibits progressively reduced enzymatic activity with increasing NaCl concentrations, retaining 40% activity at 500 mM NaCl (Yang, Zhang, Wang, Yu, & Wei, 2017).

In light of the limitations of existing lysins, the development of novel antimicrobial agents with low MICs, broad lytic spectra, robust environmental resistance, and enhanced stability is of critical importance.

### SUMMARY OF THE INVENTION

The present invention provides bacterial lysins that comprise one or a combination of proteins as set forth in SEQ ID NO: 1 to SEQ ID NO: 24.

The invention further provides methods of use of the bacterial lysins of the present invention.

To achieve the aforementioned objectives, the following technical measures are adopted: General Amino Acid Sequence Formula and Screening of Lysins:

The applicant has analyzed and identified a protease with lytic activity, characterized by the general amino acid sequence formula (SEQ ID NO: 49): Met-Ala-Lys-Thr-Gln-Ala-Glu-Ile-Asn-Lys-Arg-Leu-Asp-Ala-Tyr-Ala-Lys-Gly-Thr-Val-Asp-Ser-Pro-Tyr-Arg-X₁-Lys-Lys-Ala-Thr-Ser-Tyr-Asp-Pro-X₂-Phe-Gly-Val-Met-Glu-Ala-Gly-Ala-Ile-X₃-Ala-X₄-Gly-Tyr-X₅-His-Ala-Gln-Cys-Gln-X₆-Leu-Ile-Thr-Asp-Tyr-Val-Leu-Trp-Leu-Thr-Asp-Asn-Lys-Val-X₇-X₈-Trp-Gly-Asn-Ala-Lys-Asp-Gln-Ile-Lys-Gln-Ser-Tyr-Gly-Thr-Gly-Phe-Lys-Ile-His-Glu-Asn-Lys-Pro-Ser-Thr-Val-Pro-Lys-Lys-Gly-Trp-Ile-Ala-Val-Phe-Thr-Ser- Gly-Ser-Tyr-X₉-Gln-Trp-Gly-His-Ile-Gly-Ile-Val-Tyr-Asp-Gly-Gly-Asn-Thr-Ser-Thr-Phe-Thr-Ile-Leu-Glu-Gln-Asn-Trp-Asn-Gly-Tyr-Ala-Asn-Lys-Lys-Pro-Thr-Lys-Arg-Val-Asp-Asn-Tyr-Tyr-Gly-Leu-Thr-His-Phe-Ile-Glu-Ile-Pro-Val-Lys-Ala-Pro-Pro-Gly-Thr- Val-Ala-Gln-Ser-Ala-Pro-Asn-Leu-Ala-Gly-Ser-Arg-Ser-Tyr-Arg-Glu-Thr-Gly-Thr-Met-Thr-Val-Thr-Val-Asp-X₁₀-Leu-Asn-Val-X₁₁-Arg-Ala-Pro-Asn-Thr-Ser-Gly-Glu-Ile-Val-Ala-Val-Tyr-Lys-Arg-Gly-Glu-Ser-Phe-Asp-Tyr-Asp-Thr-Val-Ile-Ile-Asp-Val-Asn-Gly-Tyr-Val-Trp-Val-Ser-Tyr-Ile-Gly-Gly-Ser-Gly-Lys-Arg-Asn-Tyr-Val-Ala-Thr-Gly-Ala- Thr-Lys-Asp-Gly-Lys-Arg-Phe-Gly-Asn-Ala-Trp-Gly-Thr-Phe-Lys, wherein: Position 26 (X₁) is selected from Ile or Val; Position 35 (X₂) is selected from Ser or Ala; Position 45 (X₃) is selected from Asp or Ala; Position 47 (X₄) is selected from Asp or Ala; Position 50 (X₅) is selected from Tyr or Ala; Position 56 (X₆) is selected from Asp or Ala; Position 71 (X₇) is selected from Arg or Ala; Position 72 (X₈) is selected from Thr or Ala; Position 113 (X₉) is selected from Glu or Gln; Position 195 (X₁₀) is selected from Ala or Gly; and Position 199 (X₁₁) is selected from Arg or Ala.

Based on the sequence features, the applicant synthesized and screened 24 lysins capable of lysing bacteria from the genera Staphylococcus, Streptococcus, and Enterococcus. These lysins are represented as SEQ ID NO: 1 to SEQ ID NO: 24.

The protection scope of the invention encompasses one or a combination of proteins set forth in SEQ ID NO: 1 to SEQ ID NO: 24.

Among the aforementioned sequences, preferred lysins comprise one or a combination of SEQ ID NO: 2 (LLysSA9.1), SEQ ID NO: 4 (LLysSA9.2), SEQ ID NO: 7 (LLysSA9.3), SEQ ID NO: 8 (LLysSA9.4), SEQ ID NO: 9 (LLysSA9.5), SEQ ID NO: 11 (LLysSA9.6), SEQ ID NO: 12 (LLysSA9.7), SEQ ID NO: 14 (LLysSA9.8), and/or SEQ ID NO: 22 (LLysSA9.9).

The protection scope of the invention further encompasses: (a) expression vectors comprising the lysin-encoding genes, recombinant strains comprising the expression vectors, compositions comprising the lysins, and formulations of the lysins; (b) use of the the lysins, expression vectors, recombinant strains, compositions, or formulations in the preparation of bacterial inhibitors, lytic agents, or inactivators; (c) use of the the lysins, expression vectors, recombinant strains, compositions, or formulations in the preparation of a medicament for: bacterial eradication; prevention of bacterial infection; or treatment of diseases associated with bacterial infection; (d) use of the the lysins, expression vectors, recombinant strains, compositions, or formulations for in vitro inhibition or lysis of bacteria. Immune serum obtained using the aforementioned lysins.

In the methods of use of the present invention, the bacteria are preferably selected from the genera Staphylococcus, Streptococcus, and/or Enterococcus.

Specifically, the genera Staphylococcus includes: Staphylococcus aureus, Staphylococcus epidermidis, Staphylococcus capitis, Staphylococcus pseudintermedius, Staphylococcus hominis, Staphylococcus haemolyticus, Staphylococcus saprophyticus, or Staphylococcus caprae.

The genera Streptococcus includes: Streptococcus suis, Streptococcus dysgalactiae, Streptococcus agalactiae, Streptococcus pyogenes, Streptococcus mutans, or Streptococcus gallolyticus.

The genera Enterococcus includes: Enterococcus faecium or Enterococcus faecalis.

Compared to existing technology, the present invention possesses the following advantages and beneficial effects:

Moreover, the present invention provides a lysin comprising 264 amino acids. Through selective sequence optimization, the lysin of the invention has ultimately been obtained. The lysin of the present invention effectively lyses bacteria of genera Staphylococcus, Streptococcus, and/or Enterococcus while exhibiting superior stability against inactivation.

The LLysSA9.1 to LLysSA9.9 variants (corresponding to SEQ ID NOs: 2, 4, 7, 8, 9, 11, 12, 14, and 22) demonstrate MIC₅₀ values of 1-2 μg/mL and MIC₉₀ values of 2-8 μg/mL against Staphylococcus aureus, representing a 4-16-fold improvement in MIC₉₀ compared to the reported lysin CF-301. Against Staphylococcus aureus ATCC25923, USA300, and ATCC43300 strains, LLysSA9.1 to LLysSA9.9 (corresponding to SEQ ID NOs: 2, 4, 7, 8, 9, 11, 12, 14, and 22) exhibit MIC values of 0.25-2 μg/mL, 0.5-4 μg/mL, and 0.5-4 μg/mL, respectively. These values correspond to 16-120-fold, 8-64-fold, and 16-128-fold improvements over lysins LysRODI (for ATCC25923), LysK (for USA300), and CF-301 (for ATCC43300), respectively.

LLysSA9.1 to LLysSA9.9 (corresponding to SEQ ID NOs: 2, 4, 7, 8, 9, 11, 12, 14, and 22) exhibit broad-spectrum bactericidal activity against multiple Staphylococcus, Streptococcus, and/or Enterococcus species, whereas lysin LysK demonstrates activity limited to Staphylococcus strains.

LLysSA9.1 to LLysSA9.9 (corresponding to SEQ ID NOs: 2, 4, 7, 8, 9, 11, 12, 14, and 22) maintain NaCl stability, retaining 90-100% enzymatic activity at NaCl concentrations up to 500 mM. In contrast, lysin ClyF shows progressively reduced activity with increasing NaCl concentration, retaining only 40% activity at 500 mM.

LLysSA9.1 to LLysSA9.9 (corresponding to SEQ ID NOs: 2, 4, 7, 8, 9, 11, 12, 14, and 22) demonstrate pH stability within the range of pH 7-10, maintaining 90-100% enzymatic activity, whereas lysin ClyF retains only 60% activity under equivalent pH conditions.

LLysSA9.1 to LLysSA9.9 (corresponding to SEQ ID NOs: 2, 4, 7, 8, 9, 11, 12, 14, and 22) exhibit exceptional storage stability, retaining 100% enzymatic activity after 6 months at 4° C. By comparison, lysin LysRODI retains only 50% activity after 3 months under identical storage conditions.

LLysSA9.1 to LLysSA9.9 (corresponding to SEQ ID NOs: 2, 4, 7, 8, 9, 11, 12, 14, and 22) demonstrate therapeutic efficacy against both localized and systemic infections, providing a novel pharmaceutical agent for treating diseases caused by Staphylococcus, Streptococcus, and/or Enterococcus infections.

### BRIEF DESCRIPTION OF THE DRAWINGS

FIG. 1 shows preliminary determination of bactericidal activity of the lysin against Staphylococcus aureus.

FIG. 2 illustrates the structural composition of the lysin.

FIGS. 3A to 3C depict the therapeutic efficacy of LLysSA9.1 to LLysSA9.9 (corresponding to SEQ ID NOs: 2, 4, 7, 8, 9, 11, 12, 14, and 22) via local administration in various infections, where FIG. 3A shows the actual images of mouse wounds at different treatment days in a Staphylococcus aureus wound infection model following LLysSA9.1 to LLysSA9.9 therapy; FIG. 3B shows the percentage of wound size (vertical axis is Wound size (%)); and FIG. 3C shows bacterial load in blood after local administration of LLysSA9.1 to LLysSA9.9 for treating systemic Staphylococcus aureus infection (vertical axis is CFU/mL).

FIGS. 4A and 4B demonstrate the therapeutic efficacy of LLysSA9.1 to LLysSA9.9 via systemic administration in various infections, where FIG. 4A shows bacterial load in blood after systemic administration of LLysSA9.1 to LLysSA9.9 for treating systemic Staphylococcus aureus infection (vertical axis is CFU/mL); and FIG. 4B shows bacterial load in blood after systemic administration of LLysSA9.1 to LLysSA9.9 for treating systemic Streptococcus suis infection (vertical axis is CFU/mL).

### DETAILED DESCRIPTION OF THE INVENTION

The present invention employs bioinformatics methods to screen lysins, resulting in the design of 24 gene sequences encoding lysins. These sequences are expressed in Escherichia coli to produce corresponding lysins. The present invention is further illustrated below through examples; however, the scope of protection is not limited to these examples. Unless otherwise stated, the technical solutions described herein are conventional, and reagents or materials are commercially sourced.

### Example 1

### General Amino Acid Sequence Formula and Screening of Lysins

The applicant has analyzed and identified a protease with lytic activity, characterized by the general amino acid sequence formula (SEQ ID NO: 49): Met-Ala-Lys-Thr-Gln-Ala-Glu-Ile-Asn-Lys-Arg-Leu-Asp-Ala-Tyr-Ala-Lys-Gly-Thr-Val-Asp-Ser-Pro-Tyr-Arg-X₁-Lys-Lys-Ala-Thr-Ser-Tyr-Asp-Pro-X₂-Phe-Gly-Val-Met-Glu-Ala-Gly-Ala-Ile-X₃-Ala-X₄-Gly-Tyr-X₅-His-Ala-Gln-Cys-Gln-X₆-Leu-Ile-Thr-Asp-Tyr-Val-Leu-Trp-Leu-Thr-Asp-Asn-Lys-Val-X₇-X₈-Trp-Gly-Asn-Ala-Lys-Asp-Gln-Ile-Lys-Gln-Ser-Tyr-Gly-Thr-Gly-Phe-Lys-Ile-His-Glu-Asn-Lys-Pro-Ser-Thr-Val-Pro-Lys-Lys-Gly-Trp-Ile-Ala-Val-Phe-Thr-Ser- Gly-Ser-Tyr-X₉-Gln-Trp-Gly-His-Ile-Gly-Ile-Val-Tyr-Asp-Gly-Gly-Asn-Thr-Ser-Thr-Phe-Thr-Ile-Leu-Glu-Gln-Asn-Trp-Asn-Gly-Tyr-Ala-Asn-Lys-Lys-Pro-Thr-Lys-Arg-Val-Asp-Asn-Tyr-Tyr-Gly-Leu-Thr-His-Phe-Ile-Glu-Ile-Pro-Val-Lys-Ala-Pro-Pro-Gly-Thr- Val-Ala-Gln-Ser-Ala-Pro-Asn-Leu-Ala-Gly-Ser-Arg-Ser-Tyr-Arg-Glu-Thr-Gly-Thr-Met-Thr-Val-Thr-Val-Asp-X₁₀-Leu-Asn-Val-X₁₁-Arg-Ala-Pro-Asn-Thr-Ser-Gly-Glu-Ile-Val-Ala-Val-Tyr-Lys-Arg-Gly-Glu-Ser-Phe-Asp-Tyr-Asp-Thr-Val-Ile-Ile-Asp-Val-Asn-Gly-Tyr-Val-Trp-Val-Ser-Tyr-Ile-Gly-Gly-Ser-Gly-Lys-Arg-Asn-Tyr-Val-Ala-Thr-Gly-Ala- Thr-Lys-Asp-Gly-Lys-Arg-Phe-Gly-Asn-Ala-Trp-Gly-Thr-Phe-Lys, wherein: Position 26 (X₁) is selected from Ile or Val; Position 35 (X₂) is selected from Ser or Ala; Position 45 (X₃) is selected from Asp or Ala; Position 47 (X₄) is selected from Asp or Ala; Position 50 (X₅) is selected from Tyr or Ala; Position 56 (X₆) is selected from Asp or Ala; Position 71 (X₇) is selected from Arg or Ala; Position 72 (X₈) is selected from Thr or Ala; Position 113 (X₉) is selected from Glu or Gln; Position 195 (X₁₀) is selected from Ala or Gly; and Position 199 (X₁₁) is selected from Arg or Ala.

Based on the sequence features, the applicant synthesized and screened 24 lysins capable of lysing Staphylococcus, Streptococcus, and related bacterial genera. Additional lysins may be derived by exploring variations within the above general formula. Positional variations of the lysins are summarized in TABLE 1, with sequences provided as SEQ ID NO: 1 to SEQ ID NO: 24.

TABLE 1
Summary of 24 lysin sequences with antibacterial activity

| SEQ | | | | | | | | | | | | |
|---|---|---|---|---|---|---|---|---|---|---|---|---|
| ID | Name of | Position | Position | Position | Position | Position | Position | Position | Position | Position | Position | Position |
| NO: | lysin | X₁ | X₂ | X₃ | X₄ | X₅ | X₆ | X₇ | X₈ | X₉ | X₁₀ | X₁₁ |
| 1 | LLysSA0.1 | Ile | Ser | Ala | Ala | Tyr | Ala | Arg | Thr | Gln | Ala | Arg |
| 2* | LLysSA9.1 | Val | Ser | Asp | Asp | Tyr | Asp | Arg | Thr | Gln | Ala | Arg |
| 3 | LLysSA0.2 | Ile | Ser | Asp | Asp | Tyr | Asp | Arg | Thr | Glu | Ala | Arg |
| 4* | LLysSA9.2 | Ile | Ala | Asp | Asp | Tyr | Asp | Arg | Thr | Gln | Ala | Arg |
| 5 | LLysSA0.3 | Val | Ser | Asp | Asp | Tyr | Asp | Arg | Thr | Glu | Ala | Arg |
| 6 | LLysSA0.4 | Ile | Ala | Ala | Ala | Ala | Ala | Ala | Ala | Gln | Gly | Ala |
| 7* | LLysSA9.3 | Ile | Ser | Asp | Asp | Tyr | Asp | Arg | Thr | Gln | Ala | Ala |
| 8* | LLysSA9.4 | Ile | Ser | Asp | Asp | Tyr | Asp | Arg | Thr | Gln | Gly | Ala |
| 9* | LLysSA9.5 | Ile | Ser | Asp | Asp | Ala | Asp | Arg | Thr | Gln | Ala | Arg |
| 10 | LLysSA0.5 | Ile | Ala | Ala | Asp | Tyr | Asp | Arg | Thr | Gln | Gly | Arg |
| 11* | LLysSA9.6 | Ile | Ser | Asp | Asp | Tyr | Asp | Ala | Thr | Gln | Ala | Arg |
| 12* | LLysSA9.7 | Ile | Ser | Asp | Asp | Tyr | Asp | Arg | Thr | Gln | Ala | Arg |
| 13 | LLysSA0.6 | Ile | Ser | Asp | Asp | Ala | Asp | Ala | Ala | Gln | Ala | Arg |
| 14* | LLysSA9.8 | Ile | Ser | Asp | Asp | Tyr | Asp | Arg | Ala | Gln | Ala | Arg |
| 15 | LLysSA0.7 | Ile | Ala | Asp | Asp | Ala | Asp | Ala | Thr | Gln | Ala | Arg |
| 16 | LLysSA0.8 | Ile | Ala | Asp | Asp | Ala | Asp | Arg | Ala | Gln | Ala | Arg |
| 17 | LLysSA0.9 | Ile | Ala | Asp | Asp | Tyr | Asp | Ala | Ala | Gln | Ala | Arg |
| 18 | LLysSA0.10 | Ile | Ala | Asp | Asp | Ala | Asp | Ala | Ala | Gln | Ala | Arg |
| 19 | LLysSA0.11 | Ile | Ala | Asp | Asp | Ala | Asp | Arg | Thr | Gln | Ala | Arg |
| 20 | LLysSA0.12 | Ile | Ala | Asp | Asp | Tyr | Asp | Ala | Thr | Gln | Ala | Arg |
| 21 | LLysSA0.13 | Ile | Ala | Asp | Asp | Tyr | Asp | Arg | Ala | Gln | Ala | Arg |
| 22* | LLysSA9.9 | Ile | Ser | Asp | Asp | Ala | Asp | Ala | Thr | Gln | Ala | Arg |
| 23 | LLysSA0.14 | Ile | Ser | Asp | Asp | Ala | Asp | Arg | Ala | Gln | Ala | Arg |
| 24 | LLysSA0.15 | Ile | Ser | Asp | Asp | Tyr | Asp | Ala | Ala | Gln | Ala | Arg |

### Example 2

### Construction of Lysin Expression Vectors and Expression Purification

DNA sequences encoding the lysins were synthesized in full length by Sangon Biotech (Shanghai) Co., Ltd. and cloned into the expression vector pET-14b. The resulting constructs were then transformed into Escherichia coli BL21.

The DNA sequences comprise polynucleotides encoding the proteins of SEQ ID NO: 1 to SEQ ID NO: 24 (corresponding to SEQ ID NO: 25 to SEQ ID NO: 48, respectively) and the polynucleotide encoding CF-301 (Schuch et al., 2014).

Following induction of expression in the transformed strains, bacterial cells were harvested and lysed via high-pressure homogenization. The lysins SEQ ID NO: 1 to SEQ ID NO: 24 and CF-301 were subsequently purified using chromatography and ultra-filtration.

### Example 3

Bactericidal Activity of Lysins Against Staphylococcus aureus

Staphylococcus aureus strain ATCC29213 was cultured to the logarithmic growth phase. Bacterial suspensions from this phase were used to prepare semi-solid agar plates with a bacterial lawn as the upper layer. Crude enzyme lysates of the lysins (prepared via cell disruption) were spotted onto the plates, which were then incubated at 37° C. overnight. Formation of clear zones (plaques) indicated bactericidal activity.

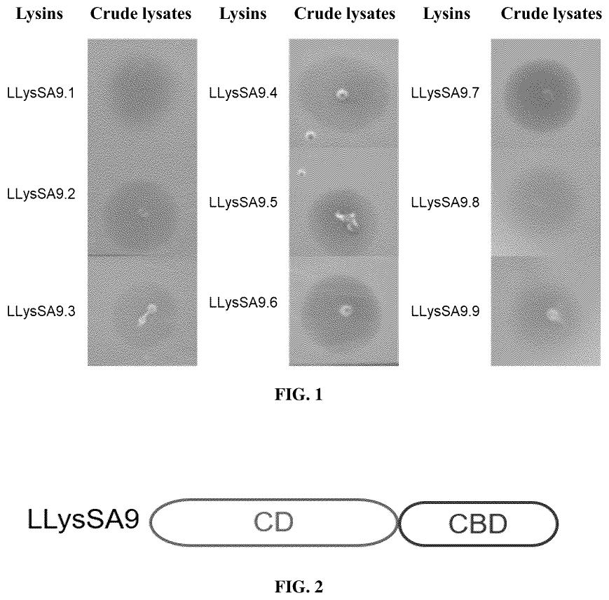

Results demonstrate that SEQ ID NO: 1 to SEQ ID NO: 24 all exhibited bactericidal activity against Staphylococcus aureus. Through optimization, lysins with superior efficacy—SEQ ID NO: 2, SEQ ID NO: 4, SEQ ID NO: 7, SEQ ID NO: 8, SEQ ID NO: 9, SEQ ID NO: 11, SEQ ID NO: 12, SEQ ID NO: 14, and SEQ ID NO: 22-were selected and designated LLysSA9.1˜LLysSA9.9, respectively. As shown in FIG. 1, crude lysates of LLysSA9.1˜LLysSA9.9 produced transparent, well-defined clear zones on the bacterial lawn, confirming their potent lytic activity against Staphylococcus aureus.

### Example 4

Minimum Inhibitory Concentration (MIC) of Lysins LLysSA9.1˜LLysSA9.9 Against Staphylococcus aureus

In accordance with the Clinical and Laboratory Standards Institute (CLSI) guidelines, the microbroth dilution method was employed to determine the MIC of the lysins. A total of 16 MSSA strains and 50 MRSA strains were tested. The MIC₅₀ and MIC₉₀ values for the lysins against all 66 Staphylococcus aureus strains were subsequently calculated. ATCC strains were sourced from the American Type Culture Collection (ATCC), while other strains were selected from published literature (Zou et al., 2022). Results are as follows:

TABLE 2
MICs of lysins against selected Staphylococcus aureus strains

| | | LLysSA9.1 | LLysSA9.2 | LLysSA9.3 | LLysSA9.4 | LLysSA9.5 |
|---|---|---|---|---|---|---|
| Strain type | Strain No. | MIC (μg/mL) | | | | |
| MIC₅₀ | | 1 | 1 | 2 | 1 | 1 |
| MIC₉₀ | | 4 | 4 | 4 | 4 | 8 |
| Methicillin- | ATCC6538 | 16 | 16 | 1 | 2 | 0.5 |
| sensitive | ATCC29213 | 4 | 4 | 0.5 | 0.5 | 8 |
| Staphylococcus | ATCC25923 | 1 | 0.5 | 1 | 1 | 1 |
| aureus | RN4220 | 1 | 1 | 16 | 0.5 | 1 |
| (MSSA) | SQNPS | 4 | 1 | 0.5 | 1 | 4 |
| | LSA684 | 0.5 | 2 | 2 | 1 | 8 |
| | LSA866 | 0.25 | 4 | 1 | 1 | 0.5 |
| Methicillin- | USA300 | 1 | 0.5 | 4 | 4 | 1 |
| resistant | ATCC43300 | 0.5 | 0.5 | 1 | 1 | 2 |
| Staphylococcus | LSA225 | 1 | 0.25 | 4 | 0.5 | 2 |
| aureus | LSA522 | 2 | 1 | 2 | 0.5 | 2 |
| (MRSA) | LSA592 | 4 | 4 | 2 | 1 | 1 |
| | LSA608 | 1 | 0.5 | 4 | 16 | 8 |
| | LSA625 | 0.5 | 1 | 1 | 1 | 0.5 |
| | LSA546 | 2 | 8 | 0.5 | 2 | 1 |
| | LSA710 | 2 | 1 | 2 | 1 | 1 |
| | LSA726 | 0.5 | 4 | 0.5 | 2 | 1 |
| | LSA668 | 2 | 2 | 2 | 1 | 0.5 |
| | LSA745 | 0.5 | 1 | 0.25 | 2 | 2 |
| | LSA753 | 4 | 0.5 | 2 | 2 | 2 |
| | LSA754 | 8 | 1 | 1 | 4 | 1 |
| | LSA775 | 2 | 2 | 2 | 0.5 | 2 |
| | LSA785 | 1 | 2 | 8 | 0.25 | 1 |
| | LSA795 | 1 | 1 | 2 | 4 | 4 |

| | | | | | | Lysin |
|---|---|---|---|---|---|---|
| | | LLysSA9.6 | LLysSA9.7 | LLysSA9.8 | LLysSA9.9 | CF-301 |
| Strain type | Strain No. | MIC (μg/mL) | | | | |
| MIC₅₀ | | 2 | 1 | 1 | 2 | 16 |
| MIC₉₀ | | 8 | 2 | 4 | 4 | 32 |
| Methicillin- | ATCC6538 | 8 | 0.5 | 0.5 | 1 | 32 |
| sensitive | ATCC29213 | 2 | 1 | 0.5 | 16 | 16 |
| Staphylococcus | ATCC25923 | 0.25 | 1 | 2 | 0.5 | 8 |
| aureus | RN4220 | 0.5 | 1 | 4 | 1 | 4 |
| (MSSA) | SQNPS | 8 | 1 | 1 | 2 | 4 |
| | LSA684 | 1 | 0.5 | 16 | 2 | 16 |
| | LSA866 | 2 | 0.5 | 0.5 | 4 | 16 |
| Methicillin- | USA300 | 1 | 2 | 0.5 | 2 | 8 |
| resistant | ATCC43300 | 1 | 4 | 1 | 0.5 | 32 |
| Staphylococcus | LSA225 | 0.5 | 1 | 1 | 0.5 | 16 |
| aureus | LSA522 | 0.5 | 0.5 | 0.25 | 1 | 32 |
| (MRSA) | LSA592 | 1 | 2 | 2 | 4 | 32 |
| | LSA608 | 8 | 1 | 2 | 0.5 | 8 |
| | LSA625 | 1 | 1 | 1 | 4 | 16 |
| | LSA546 | 2 | 2 | 1 | 1 | 16 |
| | LSA710 | 4 | 2 | 2 | 8 | 128 |
| | LSA726 | 2 | 2 | 4 | 4 | 32 |
| | LSA668 | 2 | 2 | 2 | 2 | 16 |
| | LSA745 | 1 | 0.5 | 0.5 | 2 | 8 |
| | LSA753 | 2 | 16 | 1 | 0.5 | 64 |
| | LSA754 | 1 | 1 | 1 | 1 | 16 |
| | LSA775 | 4 | 1 | 1 | 2 | 16 |
| | LSA785 | 4 | 0.25 | 1 | 2 | 4 |
| | LSA795 | 8 | 2 | 16 | 1 | 32 |

The invention tested 66 strains in total. LLysSA9.1˜LLysSA9.9 exhibited MIC₅₀ values of 1-2 μg/mL and MIC₉₀ values of 2-8 μg/mL, with representative results shown in TABLE 2. The MIC₉₀ values of LLysSA9.1˜LLysSA9.9 were 4-16-fold lower than those reported for lysin CF-301 (Indiani et al., 2019).

### Example 5

### Lytic Spectra of LLysSA9.1˜LLysSA9.9 Against Staphylococcus, Streptococcus, and Enterococcus

A total of 66 Staphylococcus aureus strains (Zou et al., 2022) were randomly selected from publicly available databases, including antibiotic-susceptible strains (ATCC29213, ATCC6538) and strains with diverse resistance phenotypes (ATCC25923, ATCC8095, RN4220, SQNPS, USA300, ATCC43300, LSA555, LSA612, LSA531, LSA795, etc.). Additional tested strains comprised: 5 Staphylococcus epidermidis, 6 Staphylococcus capitis, 1 Staphylococcus pseudintermedius, 3 Staphylococcus hominis, 3 Staphylococcus haemolyticus, 1 Staphylococcus saprophyticus, 1 Staphylococcus caprae; 21 Streptococcus suis strains (Dong et al., 2021), including antibiotic-susceptible strain P1/7 and strains harboring resistance genes (SC19, LSM102, LSM178, LSSP237, etc.); 2 Streptococcus dysgalactiae; 3 Streptococcus agalactiae; 2 Streptococcus pyogenes; 1 Streptococcus mutans; 1 Streptococcus gallolyticus; 1 Enterococcus faecium; 2 Enterococcus faecalis. Bactericidal activity of the lysins against the above strains was assessed as follows:

Strains were cultured to the logarithmic growth phase. Bacterial suspensions were centrifuged, washed, and resuspended in an equal volume of buffer, then diluted to the desired concentration. Diluted bacterial suspension were mixed with a defined quantity of lysin and incubated at 37° C. with 200 rpm shaking for 1 hour. Reaction mixtures were serially diluted and plated for bacterial enumeration via plate counting. A mixture of buffer and bacterial suspension served as the control group.

TABLE 3
Analysis of lytic spectra of the lysins

| | Strain | Quantity of strains that can be lysed | | | | |
|---|---|---|---|---|---|---|
| Strain type | quantity | LLysSA9.1 | LLysSA9.2 | LLysSA9.3 | LLysSA9.4 | LLysSA9.5 |
| Staphylococcus | 66 | 66 | 66 | 66 | 66 | 66 |
| aureus | | | | | | |
| Staphylococcus | 5 | 5 | 5 | 5 | 5 | 5 |
| epidermidis | | | | | | |
| Staphylococcus | 6 | 6 | 6 | 6 | 6 | 6 |
| capitis | | | | | | |
| Staphylococcus | 1 | 1 | 1 | 1 | 1 | 1 |
| pseudintermedius | | | | | | |
| Staphylococcus | 3 | 3 | 3 | 3 | 3 | 3 |
| hominis | | | | | | |
| Staphylococcus | 3 | 3 | 3 | 3 | 3 | 3 |
| haemolyticus | | | | | | |
| Staphylococcus | 1 | 1 | 1 | 1 | 1 | 1 |
| saprophyticus | | | | | | |
| Staphylococcus | 1 | 1 | 1 | 1 | 1 | 1 |
| caprae | | | | | | |
| Streptococcus | 21 | 21 | 21 | 21 | 21 | 21 |
| suis | | | | | | |
| Streptococcus | 2 | 2 | 2 | 2 | 2 | 2 |
| dysgalactiae | | | | | | |
| Streptococcus | 3 | 3 | 3 | 3 | 3 | 3 |
| agalactiae | | | | | | |
| Streptococcus | 2 | 2 | 2 | 2 | 2 | 2 |
| pyogenes | | | | | | |
| Streptococcus | 1 | 1 | 1 | 1 | 1 | 1 |
| mutans | | | | | | |
| Streptococcus | 1 | 1 | 1 | 1 | 1 | 1 |
| gallolyticus | | | | | | |
| Enterococcus | 1 | 1 | 1 | 1 | 1 | 1 |
| faecium | | | | | | |
| Enterococcus | 2 | 2 | 2 | 2 | 2 | 2 |
| faecalis | | | | | | |

| | Quantity of strains that can be lysed | | | | |
|---|---|---|---|---|---|
| Strain type | LLysSA9.6 | LLysSA9.7 | LLysSA9.8 | LLysSA9.9 | CF-301 |
| Staphylococcus | 66 | 66 | 66 | 66 | 66 |
| aureus | | | | | |
| Staphylococcus | 5 | 5 | 5 | 5 | 4 |
| epidermidis | | | | | |
| Staphylococcus | 6 | 6 | 6 | 6 | 6 |
| capitis | | | | | |
| Staphylococcus | 1 | 1 | 1 | 1 | 1 |
| pseudintermedius | | | | | |
| Staphylococcus | 3 | 3 | 3 | 3 | 2 |
| hominis | | | | | |
| Staphylococcus | 3 | 3 | 3 | 3 | 3 |
| haemolyticus | | | | | |
| Staphylococcus | 1 | 1 | 1 | 1 | 1 |
| saprophyticus | | | | | |
| Staphylococcus | 1 | 1 | 1 | 1 | 1 |
| caprae | | | | | |
| Streptococcus | 21 | 21 | 21 | 21 | 21 |
| suis | | | | | |
| Streptococcus | 2 | 2 | 2 | 2 | 2 |
| dysgalactiae | | | | | |
| Streptococcus | 3 | 3 | 3 | 3 | 3 |
| agalactiae | | | | | |
| Streptococcus | 2 | 2 | 2 | 2 | 2 |
| pyogenes | | | | | |
| Streptococcus | 1 | 1 | 1 | 1 | 1 |
| mutans | | | | | |
| Streptococcus | 1 | 1 | 1 | 1 | 1 |
| gallolyticus | | | | | |
| Enterococcus | 1 | 1 | 1 | 1 | 1 |
| faecium | | | | | |
| Enterococcus | 2 | 2 | 2 | 2 | 2 |
| faecalis | | | | | |

As shown in Table 3, LLysSA9.1˜LLysSA9.9 exhibited 100% lysis of all Staphylococcus strains tested, along with potent lytic activity against multiple Streptococcus and Enterococcus species.

### Example 6

### Effect of pH on Bactericidal Activity of LLysSA9.1˜LLysSA9.9

Staphylococcus aureus was cultured to the logarithmic growth phase. Bacterial suspensions from this phase were centrifuged, washed, and resuspended in buffers at varying pH values. The lysins were mixed 1:1 (v/v) with the bacterial suspensions, and changes in absorbance at 600 nm (OD₆₀₀) were monitored using a microplate reader. A control group consisted of buffer mixed with bacterial suspension. After testing, the reduction in OD₆₀₀ for each group was calculated relative to the control group. The group showing the maximum OD₆₀₀ reduction was assigned a value of 1, and the relative enzymatic activity of other groups was determined by comparison.

### Preparation of Buffers at Different pH Values:

pH=7 and pH=8: Buffers were prepared by mixing 0.1 M HEPES and 0.1 M HEPES sodium salt, adjusted to pH 7 or 8, filtered through a 0.22 μm membrane, and diluted to 30 mM prior to use. pH=9 and pH=10: Buffers were prepared using 0.1 M CHES, pH-adjusted with NaOH, filtered through a 0.22 μm membrane, and diluted to 30 mM prior to use.

Results: LLysSA9.1˜LLysSA9.9 retained 91.2-100% enzymatic activity across the pH range of 7-10, with activity peaking at 100% at pH=9, 91.2% at pH=7, and 99.9% at pH=10.

### Example 7

### Effect of NaCl on Bactericidal Activity of LLysSA9.1˜LLysSA9.9

Staphylococcus aureus was cultured to the logarithmic growth phase. Bacterial suspensions from this phase were centrifuged, washed, and resuspended in NaCl solutions at varying concentrations (0 mM, 10 mM, 20 mM, 50 mM, 100 mM, 150 mM, 200 mM, and 500 mM). The lysins were mixed 1:1 (v/v) with the bacterial suspensions, and changes in absorbance at 600 nm (OD₆₀₀) were monitored using a microplate reader. A control group consisted of buffer mixed with bacterial suspension. After testing, the reduction in OD₆₀₀ for each group was calculated relative to the control group. The group showing the maximum OD₆₀₀ reduction was assigned a value of 1, and the relative enzymatic activity of other groups was determined by comparison.

Results: LLysSA9.1˜LLysSA9.9 retained 90.3-100% enzymatic activity across NaCl concentrations of 0-500 mM. Enzymatic activity peaked at 100% in the range of 150-500 mM NaCl, while activity at 0 mM NaCl was 90.3%.

### Example 8

### Sequence Analysis of LLysSA9.1˜LLysSA9.9

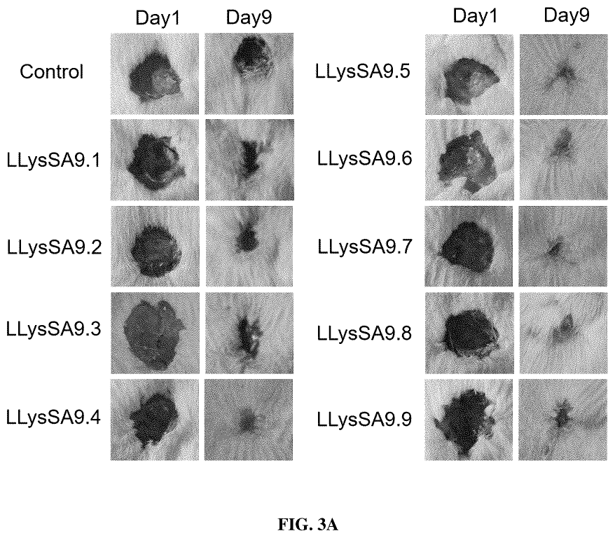

Conserved domain prediction via NCBI revealed that LLysSA9.1˜LLysSA9.9 structurally comprise an N-terminal catalytic domain (CD) and a C-terminal binding domain (CBD), as illustrated in FIG. 2.

Lysins with CDs similar to LLysSA9.1˜LLysSA9.9 include: ClyRODI-Lyso: MIC₅₀ and MIC₉₀ values >14.54 μM (438.96 μg/mL) against Staphylococcus aureus (Gutiérrez et al., 2021); ClyRODI-H5: MIC₅₀ and MIC₉₀ values=1.84 μM (64.14 μg/mL) against Staphylococcus aureus (Gutiérrez et al., 2021); LysRODI: MIC₅₀ and MIC₉₀ values=1.15 μM (63.03 μg/mL) against Staphylococcus aureus (Gutiérrez et al., 2020). In contrast, LLysSA9.1˜LLysSA9.9 exhibit MIC₅₀ values of 1-2 μg/mL and MIC₉₀ values of 2-8 μg/mL against Staphylococcus aureus, with broad-spectrum lytic activity against multiple Staphylococcus, Streptococcus, and Enterococcus species. The MIC₉₀ values of LLysSA9.1˜LLysSA9.9 are 54-219-fold, 8-32-fold, and 8-32-fold lower than those of ClyRODI-Lyso, ClyRODI-H5, and LysRODI, respectively. These results indicate that similarity or identity in the CD alone is not the primary determinant of high lytic activity; instead, the overall sequence and structural configuration of LLysSA9.1˜LLysSA9.9 are critical for their superior performance.

Lysins with CBDs similar to LLysSA9.1˜LLysSA9.9 include: CF-301: MIC₉₀=32 μg/mL against Staphylococcus aureus (Indiani et al., 2019), retaining 40% enzymatic activity at 150 mM NaCl (U.S. Pat. No. 9,034,322 B2); ClyF: Enzymatic activity progressively decreases with increasing NaCl concentration, retaining 40% activity at 500 mM NaCl (Yang, Zhang, Wang, Yu, & Wei, 2017). In contrast, LLysSA9.1˜LLysSA9.9 retain 90-100% enzymatic activity across NaCl concentrations of 0-500 mM and pH ranges of 7-10. Thus, LLysSA9.1˜LLysSA9.9 demonstrate superior adaptability to varying NaCl concentrations compared to CF-301 and ClyF. These results indicate that similarity or identity in the CBD alone is not the primary determinant of high lytic activity across diverse environments; instead, the overall sequence and structural configuration of LLysSA9.1˜LLysSA9.9 are critical for their superior performance.

### Example 9

### Use of LLysSA9.1˜LLysSA9.9 in Treating Infections Via Local Administration

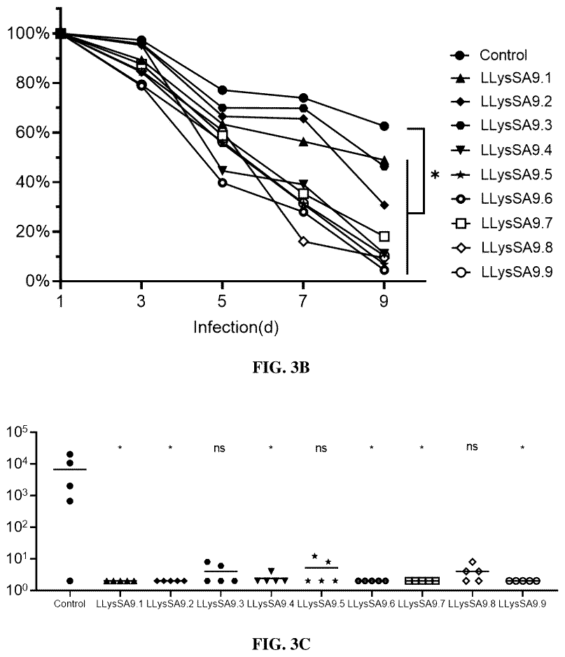

Female KM mice (6 weeks old) were used. A circular wound (1 cm diameter) was created on the dorsal surface of each mouse using a biopsy punch. Staphylococcus aureus USA300 strains (1×10⁸ CFU/mouse) were injected subcutaneously near the wound. After confirmed infection, mice were randomly divided into 10 groups (5 mice/group). Treatment groups received 10 mg/kg LLysSA9.1˜LLysSA9.9 locally on days 1, 3, 5, and 7 post-infection, while control groups received an equal volume of sterile PBS buffer. Wound images were captured on days 1 and 9 post-infection. Statistical significance (Mann-Whitney test) between treatment and control groups is annotated in Panel B of FIG. 3.

Key results of LLysSA9.1˜LLysSA9.9 in staphylococcal wound infection are shown in TABLE 4/Panel A of FIG. 3. Columns represent time points, rows denote treatment groups, and values indicate mean wound size (5 mice/group). Results demonstrate accelerated wound healing in lysin-treated groups.

TABLE 4
Efficacy of locally administered LLysSA9.1~LLysSA9.9 in a staphylococcal wound infection model

| | Control | LLysSA9.1 | LLysSA9.2 | LLysSA9.3 | LLysSA9.4 | LLysSA9.5 | LLysSA9.6 | LLysSA9.7 | LLysSA9.8 | LLysSA9.9 |
|---|---|---|---|---|---|---|---|---|---|---|
| Day | Wound size (%) | | | | | | | | | |
| 1 | 100.0 | 100.0 | 100.0 | 100.0 | 100.0 | 100.0 | 100.0 | 100.0 | 100.0 | 100.0 |
| 3 | 97.3 | 89.3 | 95.5 | 95.8 | 95.3 | 84.4 | 78.8 | 87.6 | 84.8 | 79.3 |
| 5 | 77.1 | 63.4 | 66.5 | 70.0 | 44.6 | 55.6 | 39.8 | 58.7 | 60.8 | 56.3 |
| 7 | 74.0 | 56.4 | 65.6 | 69.8 | 39.0 | 31.2 | 28.0 | 35.3 | 16.1 | 31.5 |
| 9 | 62.6 | 48.9 | 30.7 | 46.4 | 11.2 | 6.8 | 4.5 | 18.1 | 9.3 | 10.4 |

Systemic Staphylococcus aureus Infection Model: Female Balb/c mice (6 weeks old) were used. A circular wound (1 cm diameter) was created on the dorsal surface of each mouse using a biopsy punch. Staphylococcus aureus USA300 strains (5×10⁸ CFU/mouse) were injected subcutaneously near the wound. After confirmed infection, mice were randomly divided into 10 groups (5 mice/group). Treatment groups received 10 mg/kg LLysSA9.1˜LLysSA9.9 locally on days 1, 3, 5, and 7 post-infection, while control groups received an equal volume of sterile PBS. Blood was collected to quantify bacterial load. Data in TABLE 5 represent mean bacterial load in blood±standard deviation (SD) (5 mice/group). Statistical significance (Mann-Whitney test) between treatment and control groups is annotated in Panel C of FIG. 3.

As shown in TABLE 5/Panel C of FIG. 3A, LLysSA9.1˜LLysSA9.9 efficiently reduced bacterial load in blood, demonstrating therapeutic efficacy against bacteremia. These results demonstrate that the lysins effectively treat localized and systemic infections caused by Staphylococcus, Streptococcus, and/or Enterococcus pathogens, accelerating recovery.

TABLE 5
Efficacy of locally administered LLysSA9.1~LLysSA9.9 in a staphylococcal systemic infection model

| | | | | | | | | | |
|---|---|---|---|---|---|---|---|---|---|
| Control | LLysSA9.1 | LLysSA9.2 | LLysSA9.3 | LLysSA9.4 | LLysSA9.5 | LLysSA9.6 | LLysSA9.7 | LLysSA9.8 | LLysSA9.9 |

Bacterial load in blood Log₁₀(CFU/mL) ± standard deviation (SD)

| | | | | | | | | | |
|---|---|---|---|---|---|---|---|---|---|
| 3.82 ± | 0.00 ± | 0.00 ± | 0.53 ± | 0.20 ± | 0.66 ± | 0.00 ± | 0.00 ± | 0.56 ± | 0.00 ± |
| 1.54 | 0.00 | 0.00 | 0.41 | 0.24 | 0.49 | 0.00 | 0.00 | 0.36 | 0.00 |

### Example 10

### Use of LLysSA9.1˜LLysSA9.9 in Treating Infections Via Systemic Administration

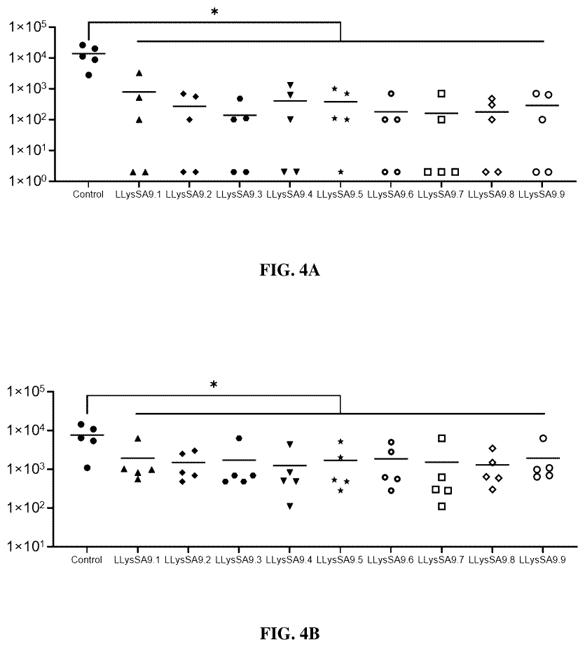

Systemic Staphylococcus aureus Infection Model: Female KM mice (5 weeks old) were used. Staphylococcus aureus USA300 strains (2×10⁸ CFU/mouse) were intravenously injected into the mice. After 1.5 hours, mice were randomized into 10 groups (5 mice/group). Treatment groups received 10 mg/kg LLysSA9.1˜LLysSA9.9 via intravenous injection, while control groups received an equal volume of sterile PBS. Blood was collected 96 hours post-treatment to quantify bacterial load. Data in TABLE 6 represent mean bacterial load in blood±standard deviation (SD) (5 mice/group). Statistical significance (Mann-Whitney test) between treatment and control groups is annotated in Panel A of FIG. 4.

As shown in TABLE 6/Panel A of FIG. 4, LLysSA9.1˜LLysSA9.9 significantly reduced bacterial load in blood, demonstrating therapeutic efficacy against bacteremia.

TABLE 6
Efficacy of systemically administered LLysSA9.1~LLysSA9.9 in a staphylococcal systemic infection model

| | | | | | | | | | |
|---|---|---|---|---|---|---|---|---|---|
| Control | LLysSA9.1 | LLysSA9.2 | LLysSA9.3 | LLysSA9.4 | LLysSA9.5 | LLysSA9.6 | LLysSA9.7 | LLysSA9.8 | LLysSA9.9 |

Bacterial load in blood Log₁₀(CFU/mL) ± standard deviation (SD)

| | | | | | | | | | |
|---|---|---|---|---|---|---|---|---|---|
| 4.14 ± | 2.90 ± | 2.43 ± | 2.14 ± | 2.61 ± | 2.58 ± | 2.26 ± | 2.21 ± | 2.25 ± | 2.46 ± |
| 0.34 | 1.29 | 1.13 | 0.98 | 1.20 | 0.96 | 1.02 | 1.07 | 1.05 | 1.14 |

Systemic Streptococcus suis Infection Model: Female Balb/c mice (6 weeks old) were used. Streptococcus suis SC19 strains (6×10⁷ CFU/mouse) were intraperitoneally injected into the mice. After 3 hours, mice were randomly divided into 10 groups (5 mice/group). Treatment groups received 20 mg/kg LLysSA9.1˜LLysSA9.9 via intraperitoneal injection, while control groups received an equal volume of sterile PBS. Blood was collected 24 hours post-treatment to quantify bacterial load. Data in TABLE 7 represent mean bacterial load in blood±standard deviation (SD) (5 mice/group). Statistical significance (Mann-Whitney test) between treatment and control groups is annotated in Panel B of FIG. 4.

As shown in TABLE 7/Panel B of FIG. 4, LLysSA9.1˜LLysSA9.9 significantly reduced bacterial load in blood, demonstrating therapeutic efficacy against bacteremia. These results demonstrate that systemic administration of the lysins effectively treats localized and systemic infections caused by Staphylococcus, Streptococcus, and/or Enterococcus pathogens, accelerating recovery.

TABLE 7
Efficacy of systemically administered LLysSA9.1~LLysSA9.9 in a streptococcal systemic infection model

| | | | | | | | | | |
|---|---|---|---|---|---|---|---|---|---|
| Control | LLysSA9.1 | LLysSA9.2 | LLysSA9.3 | LLysSA9.4 | LLysSA9.5 | LLysSA9.6 | LLysSA9.7 | LLysSA9.8 | LLysSA9.9 |

Bacterial load in blood Log₁₀(CFU/mL) ± standard deviation (SD)

| | | | | | | | | | |
|---|---|---|---|---|---|---|---|---|---|
| 3.88 ± | 3.29 ± | 3.18 ± | 3.24 ± | 3.10 ± | 3.23 ± | 3.27 ± | 3.18 ± | 3.11 ± | 3.29 ± |
| 0.39 | 0.36 | 0.32 | 0.42 | 0.51 | 0.46 | 0.47 | 0.59 | 0.37 | 0.36 |

### Example 11

### Immune Serum of LLysSA9.1˜LLysSA9.9 Enhances Bactericidal Activity of Lysins

To evaluate whether neutralizing antibodies generated by repeated intravenous administration would impair lysin activity, Sprague-Dawley (SD) rats were intravenously injected with LLysSA9.1˜LLysSA9.9 on days 1, 2, and 7. Immune serum was harvested on day 21. The prepared immune serum was mixed with 50 μg/mL LLysSA9.1˜LLysSA9.9 and incubated for 1 hour, followed by co-incubation with an equal volume of Staphylococcus aureus USA300 (10⁹ CFU/mL). At designated time points, reaction mixtures were serially diluted, and bacterial counts were determined via plate counting. A mixture of buffer and bacterial suspension served as the negative control, while a mixture of lysin and bacterial suspension served as an additional control.

Bactericidal activity was calculated as the percentage of bacteria killed by the lysin under test conditions relative to the negative control.

Co-incubation of lysins with bacteria reduced bacterial load by 3.29±0.17 log₁₀ CFU/mL. When 20% immune serum (neutralizing antibody titer: 1:25,600) was added to the system, bacterial reduction increased to 5.30±0.21 log₁₀ CFU/mL. These results demonstrate that neutralizing antibodies do not inhibit the bactericidal activity of LLysSA9.1˜LLysSA9.9; instead, the lysins' activity in the presence of immune serum increased by 20.18-30.75%.

### Example 12

### Hyperimmune Serum of LLysSA9.1˜LLysSA9.9 Enhances Bactericidal Activity of Lysins

To evaluate whether neutralizing antibodies generated by repeated high-dose administration would impair lysin activity, Sprague-Dawley (SD) rats were intravenously injected with LLysSA9.1˜LLysSA9.9 on days 1, 2, and 7, followed by two subcutaneous multi-site injections of the LLysSA9.1˜LLysSA9.9 at 14-day intervals. Hyperimmune serum was harvested 14 days after the second subcutaneous injection. The prepared hyperimmune serum was mixed with 50 μg/mL LLysSA9.1˜LLysSA9.9 and incubated for 1 hour, followed by co-incubation with an equal volume of Staphylococcus aureus USA300 (10⁹ CFU/mL). At designated time points, reaction mixtures were serially diluted, and bacterial counts were determined via plate counting. A mixture of buffer and bacterial suspension served as the negative control, while a mixture of lysin and bacterial suspension served as an additional control.

Bactericidal activity was calculated as the percentage of bacteria killed by the lysin under test conditions relative to the negative control.

Co-incubation of lysins with bacteria reduced bacterial load by 3.29±0.17 log₁₀ CFU/mL. When 20% hyperimmune serum (neutralizing antibody titer: 1:409,600) was added to the system, bacterial reduction increased to 5.04±0.07 log₁₀ CFU/mL. These results demonstrate that neutralizing antibodies do not inhibit the bactericidal activity of LLysSA9.1˜LLysSA9.9; instead, the lysins' activity in the presence of hyperimmune serum increased by 18.31-22.99%.

### Example 13

### Storage Stability of LLysSA9.1˜LLysSA9.9

In accordance with the Clinical and Laboratory Standards Institute (CLSI) guidelines, the microbroth dilution method was employed to determine the minimum inhibitory concentration (MIC) of the lysins after 6 months of storage at 4° C. and −80° C. Staphylococcus aureus ATCC29213 and USA300 strains were selected for testing.

The MIC values of the lysins after 6 months of storage at 4° C. and −80° C. remained consistent with initial MIC values.

TABLE 8
Storage Stability of LLysSA9.1~LLysSA9.9

| | MIC(μg/mL)-Day 1 | | | | MIC(μg/mL)-Month 6 | | | |
|---|---|---|---|---|---|---|---|---|
| | Temperature | | | | | | | |
| | 4° C. | | −80° C. | | 4° C. | | −80° C. | |
| Strain | ATCC29213 | USA300 | ATCC29213 | USA300 | ATCC29213 | USA300 | ATCC29213 | USA300 |
| LLysSA9.1 | 4 | 1 | 4 | 1 | 4 | 1 | 4 | 1 |
| LLysSA9.2 | 4 | 0.5 | 4 | 0.5 | 4 | 0.5 | 4 | 0.5 |
| LLysSA9.3 | 0.5 | 4 | 0.5 | 4 | 0.5 | 4 | 0.5 | 4 |
| LLysSA9.4 | 0.5 | 4 | 0.5 | 4 | 0.5 | 4 | 0.5 | 4 |
| LLysSA9.5 | 8 | 1 | 8 | 1 | 8 | 1 | 8 | 1 |
| LLysSA9.6 | 2 | 1 | 2 | 1 | 2 | 1 | 2 | 1 |
| LLysSA9.7 | 1 | 2 | 1 | 2 | 1 | 2 | 1 | 2 |
| LLysSA9.8 | 0.5 | 0.5 | 0.5 | 0.5 | 0.5 | 0.5 | 0.5 | 0.5 |
| LLysSA9.9 | 16 | 2 | 16 | 2 | 16 | 2 | 16 | 2 |

SEQ ID NO: 1
MAKTQAEINKRLDAYAKGTVDSPYRIKKATSYDPSFGVMEAGAIAAAGYYHAQ
CQALITDYVLWLTDNKVRTWGNAKDQIKQSYGTGFKIHENKPSTVPKKGWIAVFTSGSY
QQWGHIGIVYDGGNTSTFTILEQNWNGYANKKPTKRVDNYYGLTHFIEIPVKAPPGTVA
QSAPNLAGSRSYRETGTMTVTVDALNVRRAPNTSGEIVAVYKRGESFDYDTVIIDVNGY
VWVSYIGGSGKRNYVATGATKDGKRFGNAWGTFK
SEQ ID NO: 2
MAKTQAEINKRLDAYAKGTVDSPYRVKKATSYDPSFGVMEAGAIDADGYYHAQ
CQDLITDYVLWLTDNKVRTWGNAKDQIKQSYGTGFKIHENKPSTVPKKGWIAVFTSGSY
QQWGHIGIVYDGGNTSTFTILEQNWNGYANKKPTKRVDNYYGLTHFIEIPVKAPPGTVA
QSAPNLAGSRSYRETGTMTVTVDALNVRRAPNTSGEIVAVYKRGESFDYDTVIIDVNGY
VWVSYIGGSGKRNYVATGATKDGKRFGNAWGTFK
SEQ ID NO: 3
MAKTQAEINKRLDAYAKGTVDSPYRIKKATSYDPSFGVMEAGAIDADGYYHAQ
CQDLITDYVLWLTDNKVRTWGNAKDQIKQSYGTGFKIHENKPSTVPKKGWIAVFTSGSY
EQWGHIGIVYDGGNTSTFTILEQNWNGYANKKPTKRVDNYYGLTHFIEIPVKAPPGTVAQ
SAPNLAGSRSYRETGTMTVTVDALNVRRAPNTSGEIVAVYKRGESFDYDTVIIDVNGYV
WVSYIGGSGKRNYVATGATKDGKRFGNAWGTFK
SEQ ID NO: 4
MAKTQAEINKRLDAYAKGTVDSPYRIKKATSYDPAFGVMEAGAIDADGYYHAQ
CQDLITDYVLWLTDNKVRTWGNAKDQIKQSYGTGFKIHENKPSTVPKKGWIAVFTSGSY
QQWGHIGIVYDGGNTSTFTILEQNWNGYANKKPTKRVDNYYGLTHFIEIPVKAPPGTVA
QSAPNLAGSRSYRETGTMTVTVDALNVRRAPNTSGEIVAVYKRGESFDYDTVIIDVNGY
VWVSYIGGSGKRNYVATGATKDGKRFGNAWGTFK
SEQ ID NO: 5
MAKTQAEINKRLDAYAKGTVDSPYRVKKATSYDPSFGVMEAGAIDADGYYHAQ
CQDLITDYVLWLTDNKVRTWGNAKDQIKQSYGTGFKIHENKPSTVPKKGWIAVFTSGSY
EQWGHIGIVYDGGNTSTFTILEQNWNGYANKKPTKRVDNYYGLTHFIEIPVKAPPGTVAQ
SAPNLAGSRSYRETGTMTVTVDALNVRRAPNTSGEIVAVYKRGESFDYDTVIIDVNGYV
WVSYIGGSGKRNYVATGATKDGKRFGNAWGTFK
SEQ ID NO: 6
MAKTQAEINKRLDAYAKGTVDSPYRIKKATSYDPAFGVMEAGAIAAAGYAHAQC
QALITDYVLWLTDNKVAAWGNAKDQIKQSYGTGFKIHENKPSTVPKKGWIAVFTSGSYQ
QWGHIGIVYDGGNTSTFTILEQNWNGYANKKPTKRVDNYYGLTHFIEIPVKAPPGTVAQS
APNLAGSRSYRETGTMTVTVDGLNVARAPNTSGEIVAVYKRGESFDYDTVIIDVNGYVW
VSYIGGSGKRNYVATGATKDGKRFGNAWGTFK
SEQ ID NO: 7
MAKTQAEINKRLDAYAKGTVDSPYRIKKATSYDPSFGVMEAGAIDADGYYHAQ
CQDLITDYVLWLTDNKVRTWGNAKDQIKQSYGTGFKIHENKPSTVPKKGWIAVFTSGSY
QQWGHIGIVYDGGNTSTFTILEQNWNGYANKKPTKRVDNYYGLTHFIEIPVKAPPGTVA
QSAPNLAGSRSYRETGTMTVTVDALNVARAPNTSGEIVAVYKRGESFDYDTVIIDVNGY
VWVSYIGGSGKRNYVATGATKDGKRFGNAWGTFK
SEQ ID NO: 8
MAKTQAEINKRLDAYAKGTVDSPYRIKKATSYDPSFGVMEAGAIDADGYYHAQ
CQDLITDYVLWLTDNKVRTWGNAKDQIKQSYGTGFKIHENKPSTVPKKGWIAVFTSGSY
QQWGHIGIVYDGGNTSTFTILEQNWNGYANKKPTKRVDNYYGLTHFIEIPVKAPPGTVA
QSAPNLAGSRSYRETGTMTVTVDGLNVARAPNTSGEIVAVYKRGESFDYDTVIIDVNGY
VWVSYIGGSGKRNYVATGATKDGKRFGNAWGTFK
SEQ ID NO: 9
MAKTQAEINKRLDAYAKGTVDSPYRIKKATSYDPSFGVMEAGAIDADGYAHAQC
QDLITDYVLWLTDNKVRTWGNAKDQIKQSYGTGFKIHENKPSTVPKKGWIAVFTSGSYQ
QWGHIGIVYDGGNTSTFTILEQNWNGYANKKPTKRVDNYYGLTHFIEIPVKAPPGTVAQS
APNLAGSRSYRETGTMTVTVDALNVRRAPNTSGEIVAVYKRGESFDYDTVIIDVNGYVW
VSYIGGSGKRNYVATGATKDGKRFGNAWGTFK
SEQ ID NO: 10
MAKTQAEINKRLDAYAKGTVDSPYRIKKATSYDPAFGVMEAGAIAADGYYHAQ
CQDLITDYVLWLTDNKVRTWGNAKDQIKQSYGTGFKIHENKPSTVPKKGWIAVFTSGSY
QQWGHIGIVYDGGNTSTFTILEQNWNGYANKKPTKRVDNYYGLTHFIEIPVKAPPGTVA
QSAPNLAGSRSYRETGTMTVTVDGLNVRRAPNTSGEIVAVYKRGESFDYDTVIIDVNGY
VWVSYIGGSGKRNYVATGATKDGKRFGNAWGTFK
SEQ ID NO: 11
MAKTQAEINKRLDAYAKGTVDSPYRIKKATSYDPSFGVMEAGAIDADGYYHAQ
CQDLITDYVLWLTDNKVATWGNAKDQIKQSYGTGFKIHENKPSTVPKKGWIAVFTSGSY
QQWGHIGIVYDGGNTSTFTILEQNWNGYANKKPTKRVDNYYGLTHFIEIPVKAPPGTVA
QSAPNLAGSRSYRETGTMTVTVDALNVRRAPNTSGEIVAVYKRGESFDYDTVIIDVNGY
VWVSYIGGSGKRNYVATGATKDGKRFGNAWGTFK
SEQ ID NO: 12
MAKTQAEINKRLDAYAKGTVDSPYRIKKATSYDPSFGVMEAGAIDADGYYHAQ
CQDLITDYVLWLTDNKVRTWGNAKDQIKQSYGTGFKIHENKPSTVPKKGWIAVFTSGSY
QQWGHIGIVYDGGNTSTFTILEQNWNGYANKKPTKRVDNYYGLTHFIEIPVKAPPGTVA
QSAPNLAGSRSYRETGTMTVTVDALNVRRAPNTSGEIVAVYKRGESFDYDTVIIDVNGY
VWVSYIGGSGKRNYVATGATKDGKRFGNAWGTFK
SEQ ID NO: 13
MAKTQAEINKRLDAYAKGTVDSPYRIKKATSYDPSFGVMEAGAIDADGYAHAQC
QDLITDYVLWLTDNKVAAWGNAKDQIKQSYGTGFKIHENKPSTVPKKGWIAVFTSGSYQ
QWGHIGIVYDGGNTSTFTILEQNWNGYANKKPTKRVDNYYGLTHFIEIPVKAPPGTVAQS
APNLAGSRSYRETGTMTVTVDALNVRRAPNTSGEIVAVYKRGESFDYDTVIIDVNGYVW
VSYIGGSGKRNYVATGATKDGKRFGNAWGTFK
SEQ ID NO: 14
MAKTQAEINKRLDAYAKGTVDSPYRIKKATSYDPSFGVMEAGAIDADGYYHAQ
CQDLITDYVLWLTDNKVRAWGNAKDQIKQSYGTGFKIHENKPSTVPKKGWIAVFTSGSY
QQWGHIGIVYDGGNTSTFTILEQNWNGYANKKPTKRVDNYYGLTHFIEIPVKAPPGTVA
QSAPNLAGSRSYRETGTMTVTVDALNVRRAPNTSGEIVAVYKRGESFDYDTVIIDVNGY
VWVSYIGGSGKRNYVATGATKDGKRFGNAWGTFK
SEQ ID NO: 15
MAKTQAEINKRLDAYAKGTVDSPYRIKKATSYDPAFGVMEAGAIDADGYAHAQC
QDLITDYVLWLTDNKVATWGNAKDQIKQSYGTGFKIHENKPSTVPKKGWIAVFTSGSYQ
QWGHIGIVYDGGNTSTFTILEQNWNGYANKKPTKRVDNYYGLTHFIEIPVKAPPGTVAQS
APNLAGSRSYRETGTMTVTVDALNVRRAPNTSGEIVAVYKRGESFDYDTVIIDVNGYVW
VSYIGGSGKRNYVATGATKDGKRFGNAWGTFK
SEQ ID NO: 16
MAKTQAEINKRLDAYAKGTVDSPYRIKKATSYDPAFGVMEAGAIDADGYAHAQC
QDLITDYVLWLTDNKVRAWGNAKDQIKQSYGTGFKIHENKPSTVPKKGWIAVFTSGSYQ
QWGHIGIVYDGGNTSTFTILEQNWNGYANKKPTKRVDNYYGLTHFIEIPVKAPPGTVAQS
APNLAGSRSYRETGTMTVTVDALNVRRAPNTSGEIVAVYKRGESFDYDTVIIDVNGYVW
VSYIGGSGKRNYVATGATKDGKRFGNAWGTFK
SEQ ID NO: 17
MAKTQAEINKRLDAYAKGTVDSPYRIKKATSYDPAFGVMEAGAIDADGYYHAQ
CQDLITDYVLWLTDNKVAAWGNAKDQIKQSYGTGFKIHENKPSTVPKKGWIAVFTSGSY
QQWGHIGIVYDGGNTSTFTILEQNWNGYANKKPTKRVDNYYGLTHFIEIPVKAPPGTVA
QSAPNLAGSRSYRETGTMTVTVDALNVRRAPNTSGEIVAVYKRGESFDYDTVIIDVNGY
VWVSYIGGSGKRNYVATGATKDGKRFGNAWGTFK
SEQ ID NO: 18
MAKTQAEINKRLDAYAKGTVDSPYRIKKATSYDPAFGVMEAGAIDADGYAHAQC
QDLITDYVLWLTDNKVAAWGNAKDQIKQSYGTGFKIHENKPSTVPKKGWIAVFTSGSYQ
QWGHIGIVYDGGNTSTFTILEQNWNGYANKKPTKRVDNYYGLTHFIEIPVKAPPGTVAQS
APNLAGSRSYRETGTMTVTVDALNVRRAPNTSGEIVAVYKRGESFDYDTVIIDVNGYVW
VSYIGGSGKRNYVATGATKDGKRFGNAWGTFK
SEQ ID NO: 19
MAKTQAEINKRLDAYAKGTVDSPYRIKKATSYDPAFGVMEAGAIDADGYAHAQC
QDLITDYVLWLTDNKVRTWGNAKDQIKQSYGTGFKIHENKPSTVPKKGWIAVFTSGSYQ
QWGHIGIVYDGGNTSTFTILEQNWNGYANKKPTKRVDNYYGLTHFIEIPVKAPPGTVAQS
APNLAGSRSYRETGTMTVTVDALNVRRAPNTSGEIVAVYKRGESFDYDTVIIDVNGYVW
VSYIGGSGKRNYVATGATKDGKRFGNAWGTFK
SEQ ID NO: 20
MAKTQAEINKRLDAYAKGTVDSPYRIKKATSYDPAFGVMEAGAIDADGYYHAQ
CQDLITDYVLWLTDNKVATWGNAKDQIKQSYGTGFKIHENKPSTVPKKGWIAVFTSGSY
QQWGHIGIVYDGGNTSTFTILEQNWNGYANKKPTKRVDNYYGLTHFIEIPVKAPPGTVA
QSAPNLAGSRSYRETGTMTVTVDALNVRRAPNTSGEIVAVYKRGESFDYDTVIIDVNGY
VWVSYIGGSGKRNYVATGATKDGKRFGNAWGTFK
SEQ ID NO: 21
MAKTQAEINKRLDAYAKGTVDSPYRIKKATSYDPAFGVMEAGAIDADGYYHAQ
CQDLITDYVLWLTDNKVRAWGNAKDQIKQSYGTGFKIHENKPSTVPKKGWIAVFTSGSY
QQWGHIGIVYDGGNTSTFTILEQNWNGYANKKPTKRVDNYYGLTHFIEIPVKAPPGTVA
QSAPNLAGSRSYRETGTMTVTVDALNVRRAPNTSGEIVAVYKRGESFDYDTVIIDVNGY
VWVSYIGGSGKRNYVATGATKDGKRFGNAWGTFK
SEQ ID NO: 22
MAKTQAEINKRLDAYAKGTVDSPYRIKKATSYDPSFGVMEAGAIDADGYAHAQC
QDLITDYVLWLTDNKVATWGNAKDQIKQSYGTGFKIHENKPSTVPKKGWIAVFTSGSYQ
QWGHIGIVYDGGNTSTFTILEQNWNGYANKKPTKRVDNYYGLTHFIEIPVKAPPGTVAQS
APNLAGSRSYRETGTMTVTVDALNVRRAPNTSGEIVAVYKRGESFDYDTVIIDVNGYVW
VSYIGGSGKRNYVATGATKDGKRFGNAWGTFK
SEQ ID NO: 23
MAKTQAEINKRLDAYAKGTVDSPYRIKKATSYDPSFGVMEAGAIDADGYAHAQC
QDLITDYVLWLTDNKVRAWGNAKDQIKQSYGTGFKIHENKPSTVPKKGWIAVFTSGSYQ
QWGHIGIVYDGGNTSTFTILEQNWNGYANKKPTKRVDNYYGLTHFIEIPVKAPPGTVAQS
APNLAGSRSYRETGTMTVTVDALNVRRAPNTSGEIVAVYKRGESFDYDTVIIDVNGYVW
VSYIGGSGKRNYVATGATKDGKRFGNAWGTFK
SEQ ID NO: 24
MAKTQAEINKRLDAYAKGTVDSPYRIKKATSYDPSFGVMEAGAIDADGYYHAQ
CQDLITDYVLWLTDNKVAAWGNAKDQIKQSYGTGFKIHENKPSTVPKKGWIAVFTSGSY
QQWGHIGIVYDGGNTSTFTILEQNWNGYANKKPTKRVDNYYGLTHFIEIPVKAPPGTVA
QSAPNLAGSRSYRETGTMTVTVDALNVRRAPNTSGEIVAVYKRGESFDYDTVIIDVNGY
VWVSYIGGSGKRNYVATGATKDGKRFGNAWGTFK
SEQ ID NO: 25
ATGGCGAAAACCCAGGCGGAAATCAACAAACGTCTGGATGCGTACGCGAAAG
GTACCGTTGATAGCCCGTACCGTATCAAAAAAGCGACCTCTTACGATCCGAGCTTCGG
TGTTATGGAAGCGGGTGCGATCGCTGCGGCTGGTTACTACCACGCGCAGTGCCAGGC
TCTGATCACCGATTACGTTCTGTGGCTGACCGATAACAAAGTTCGTACCTGGGGTAAC
GCGAAAGATCAGATCAAACAGTCTTACGGTACCGGTTTCAAAATCCACGAAAACAAA
CCGTCTACCGTTCCGAAAAAAGGTTGGATCGCGGTTTTCACCAGCGGTTCTTACCAG
CAGTGGGGTCACATCGGTATCGTTTACGATGGTGGTAACACCAGCACCTTCACCATCC
TGGAACAGAACTGGAACGGTTACGCGAACAAAAAACCGACCAAACGTGTTGATAAC
TACTACGGTCTGACCCACTTCATCGAAATCCCGGTTAAAGCGCCGCCGGGTACCGTTG
CTCAGTCTGCTCCGAACCTGGCTGGTTCTCGTTCTTACCGTGAAACCGGTACCATGAC
CGTTACCGTTGACGCTCTGAACGTTCGTCGTGCTCCGAACACCTCTGGTGAAATCGTT
GCTGTTTACAAACGTGGTGAATCTTTCGACTACGACACCGTTATCATCGACGTTAACG
GTTACGTTTGGGTTTCTTACATCGGTGGTTCTGGTAAACGTAACTACGTTGCTACCGGT
GCTACCAAAGACGGTAAACGTTTCGGTAACGCTTGGGGTACCTTCAAATAA
SEQ ID NO: 26
ATGGCGAAAACCCAGGCGGAAATCAACAAACGTCTGGATGCGTACGCGAAAG
GTACCGTTGATAGCCCGTACCGTGTCAAAAAAGCGACCTCTTACGATCCGAGCTTCGG
TGTTATGGAAGCGGGTGCGATCGATGCGGATGGTTACTACCACGCGCAGTGCCAGGAT
CTGATCACCGATTACGTTCTGTGGCTGACCGATAACAAAGTTCGTACCTGGGGTAACG
CGAAAGATCAGATCAAACAGTCTTACGGTACCGGTTTCAAAATCCACGAAAACAAAC
CGTCTACCGTTCCGAAAAAAGGTTGGATCGCGGTTTTCACCAGCGGTTCTTACCAGC
AGTGGGGTCACATCGGTATCGTTTACGATGGTGGTAACACCAGCACCTTCACCATCCT
GGAACAGAACTGGAACGGTTACGCGAACAAAAAACCGACCAAACGTGTTGATAACT
ACTACGGTCTGACCCACTTCATCGAAATCCCGGTTAAAGCGCCGCCGGGTACCGTTGC
TCAGTCTGCTCCGAACCTGGCTGGTTCTCGTTCTTACCGTGAAACCGGTACCATGACC
GTTACCGTTGACGCTCTGAACGTTCGTCGTGCTCCGAACACCTCTGGTGAAATCGTTG
CTGTTTACAAACGTGGTGAATCTTTCGACTACGACACCGTTATCATCGACGTTAACGG
TTACGTTTGGGTTTCTTACATCGGTGGTTCTGGTAAACGTAACTACGTTGCTACCGGTG
CTACCAAAGACGGTAAACGTTTCGGTAACGCTTGGGGTACCTTCAAATAA
SEQ ID NO: 27
ATGGCGAAAACCCAGGCGGAAATCAACAAACGTCTGGATGCGTACGCGAAAG
GTACCGTTGATAGCCCGTACCGTATCAAAAAAGCGACCTCTTACGATCCGAGCTTCGG
TGTTATGGAAGCGGGTGCGATCGATGCGGATGGTTACTACCACGCGCAGTGCCAGGAT
CTGATCACCGATTACGTTCTGTGGCTGACCGATAACAAAGTTCGTACCTGGGGTAACG
CGAAAGATCAGATCAAACAGTCTTACGGTACCGGTTTCAAAATCCACGAAAACAAAC
CGTCTACCGTTCCGAAAAAAGGTTGGATCGCGGTTTTCACCAGCGGTTCTTACGAAC
AGTGGGGTCACATCGGTATCGTTTACGATGGTGGTAACACCAGCACCTTCACCATCCT
GGAACAGAACTGGAACGGTTACGCGAACAAAAAACCGACCAAACGTGTTGATAACT
ACTACGGTCTGACCCACTTCATCGAAATCCCGGTTAAAGCGCCGCCGGGTACCGTTGC
TCAGTCTGCTCCGAACCTGGCTGGTTCTCGTTCTTACCGTGAAACCGGTACCATGACC
GTTACCGTTGACGCTCTGAACGTTCGTCGTGCTCCGAACACCTCTGGTGAAATCGTTG
CTGTTTACAAACGTGGTGAATCTTTCGACTACGACACCGTTATCATCGACGTTAACGG
TTACGTTTGGGTTTCTTACATCGGTGGTTCTGGTAAACGTAACTACGTTGCTACCGGTG
CTACCAAAGACGGTAAACGTTTCGGTAACGCTTGGGGTACCTTCAAATAA
SEQ ID NO: 28
ATGGCGAAAACCCAGGCGGAAATCAACAAACGTCTGGATGCGTACGCGAAAG
GTACCGTTGATAGCCCGTACCGTATCAAAAAAGCGACCTCTTACGATCCGGCTTTCGG
TGTTATGGAAGCGGGTGCGATCGATGCGGATGGTTACTACCACGCGCAGTGCCAGGAT
CTGATCACCGATTACGTTCTGTGGCTGACCGATAACAAAGTTCGTACCTGGGGTAACG
CGAAAGATCAGATCAAACAGTCTTACGGTACCGGTTTCAAAATCCACGAAAACAAAC
CGTCTACCGTTCCGAAAAAAGGTTGGATCGCGGTTTTCACCAGCGGTTCTTACCAGC
AGTGGGGTCACATCGGTATCGTTTACGATGGTGGTAACACCAGCACCTTCACCATCCT
GGAACAGAACTGGAACGGTTACGCGAACAAAAAACCGACCAAACGTGTTGATAACT
ACTACGGTCTGACCCACTTCATCGAAATCCCGGTTAAAGCGCCGCCGGGTACCGTTGC
TCAGTCTGCTCCGAACCTGGCTGGTTCTCGTTCTTACCGTGAAACCGGTACCATGACC
GTTACCGTTGACGCTCTGAACGTTCGTCGTGCTCCGAACACCTCTGGTGAAATCGTTG
CTGTTTACAAACGTGGTGAATCTTTCGACTACGACACCGTTATCATCGACGTTAACGG
TTACGTTTGGGTTTCTTACATCGGTGGTTCTGGTAAACGTAACTACGTTGCTACCGGTG
CTACCAAAGACGGTAAACGTTTCGGTAACGCTTGGGGTACCTTCAAATAA
SEQ ID NO: 29
ATGGCGAAAACCCAGGCGGAAATCAACAAACGTCTGGATGCGTACGCGAAAG
GTACCGTTGATAGCCCGTACCGTGTCAAAAAAGCGACCTCTTACGATCCGAGCTTCGG
TGTTATGGAAGCGGGTGCGATCGATGCGGATGGTTACTACCACGCGCAGTGCCAGGAT
CTGATCACCGATTACGTTCTGTGGCTGACCGATAACAAAGTTCGTACCTGGGGTAACG
CGAAAGATCAGATCAAACAGTCTTACGGTACCGGTTTCAAAATCCACGAAAACAAAC
CGTCTACCGTTCCGAAAAAAGGTTGGATCGCGGTTTTCACCAGCGGTTCTTACGAAC
AGTGGGGTCACATCGGTATCGTTTACGATGGTGGTAACACCAGCACCTTCACCATCCT
GGAACAGAACTGGAACGGTTACGCGAACAAAAAACCGACCAAACGTGTTGATAACT
ACTACGGTCTGACCCACTTCATCGAAATCCCGGTTAAAGCGCCGCCGGGTACCGTTGC
TCAGTCTGCTCCGAACCTGGCTGGTTCTCGTTCTTACCGTGAAACCGGTACCATGACC
GTTACCGTTGACGCTCTGAACGTTCGTCGTGCTCCGAACACCTCTGGTGAAATCGTTG
CTGTTTACAAACGTGGTGAATCTTTCGACTACGACACCGTTATCATCGACGTTAACGG
TTACGTTTGGGTTTCTTACATCGGTGGTTCTGGTAAACGTAACTACGTTGCTACCGGTG
CTACCAAAGACGGTAAACGTTTCGGTAACGCTTGGGGTACCTTCAAATAA
SEQ ID NO: 30
ATGGCGAAAACCCAGGCGGAAATCAACAAACGTCTGGATGCGTACGCGAAAG
GTACCGTTGATAGCCCGTACCGTATCAAAAAAGCGACCTCTTACGATCCGGCTTTCGG
TGTTATGGAAGCGGGTGCGATCGCTGCGGCTGGTTACGCTCACGCGCAGTGCCAGGC
TCTGATCACCGATTACGTTCTGTGGCTGACCGATAACAAAGTTGCTGCTTGGGGTAAC
GCGAAAGATCAGATCAAACAGTCTTACGGTACCGGTTTCAAAATCCACGAAAACAAA
CCGTCTACCGTTCCGAAAAAAGGTTGGATCGCGGTTTTCACCAGCGGTTCTTACCAG
CAGTGGGGTCACATCGGTATCGTTTACGATGGTGGTAACACCAGCACCTTCACCATCC
TGGAACAGAACTGGAACGGTTACGCGAACAAAAAACCGACCAAACGTGTTGATAAC
TACTACGGTCTGACCCACTTCATCGAAATCCCGGTTAAAGCGCCGCCGGGTACCGTTG
CTCAGTCTGCTCCGAACCTGGCTGGTTCTCGTTCTTACCGTGAAACCGGTACCATGAC
CGTTACCGTTGACGGTCTGAACGTTGCTCGTGCTCCGAACACCTCTGGTGAAATCGTT
GCTGTTTACAAACGTGGTGAATCTTTCGACTACGACACCGTTATCATCGACGTTAACG
GTTACGTTTGGGTTTCTTACATCGGTGGTTCTGGTAAACGTAACTACGTTGCTACCGGT
GCTACCAAAGACGGTAAACGTTTCGGTAACGCTTGGGGTACCTTCAAATAA
SEQ ID NO: 31
ATGGCGAAAACCCAGGCGGAAATCAACAAACGTCTGGATGCGTACGCGAAAG
GTACCGTTGATAGCCCGTACCGTATCAAAAAAGCGACCTCTTACGATCCGAGCTTCGG
TGTTATGGAAGCGGGTGCGATCGATGCGGATGGTTACTACCACGCGCAGTGCCAGGAT
CTGATCACCGATTACGTTCTGTGGCTGACCGATAACAAAGTTCGTACCTGGGGTAACG
CGAAAGATCAGATCAAACAGTCTTACGGTACCGGTTTCAAAATCCACGAAAACAAAC
CGTCTACCGTTCCGAAAAAAGGTTGGATCGCGGTTTTCACCAGCGGTTCTTACCAGC
AGTGGGGTCACATCGGTATCGTTTACGATGGTGGTAACACCAGCACCTTCACCATCCT
GGAACAGAACTGGAACGGTTACGCGAACAAAAAACCGACCAAACGTGTTGATAACT
ACTACGGTCTGACCCACTTCATCGAAATCCCGGTTAAAGCGCCGCCGGGTACCGTTGC
TCAGTCTGCTCCGAACCTGGCTGGTTCTCGTTCTTACCGTGAAACCGGTACCATGACC
GTTACCGTTGACGCTCTGAACGTTGCTCGTGCTCCGAACACCTCTGGTGAAATCGTTG
CTGTTTACAAACGTGGTGAATCTTTCGACTACGACACCGTTATCATCGACGTTAACGG
TTACGTTTGGGTTTCTTACATCGGTGGTTCTGGTAAACGTAACTACGTTGCTACCGGTG
CTACCAAAGACGGTAAACGTTTCGGTAACGCTTGGGGTACCTTCAAATAA
SEQ ID NO: 32
ATGGCGAAAACCCAGGCGGAAATCAACAAACGTCTGGATGCGTACGCGAAAG
GTACCGTTGATAGCCCGTACCGTATCAAAAAAGCGACCTCTTACGATCCGAGCTTCGG
TGTTATGGAAGCGGGTGCGATCGATGCGGATGGTTACTACCACGCGCAGTGCCAGGAT
CTGATCACCGATTACGTTCTGTGGCTGACCGATAACAAAGTTCGTACCTGGGGTAACG
CGAAAGATCAGATCAAACAGTCTTACGGTACCGGTTTCAAAATCCACGAAAACAAAC
CGTCTACCGTTCCGAAAAAAGGTTGGATCGCGGTTTTCACCAGCGGTTCTTACCAGC
AGTGGGGTCACATCGGTATCGTTTACGATGGTGGTAACACCAGCACCTTCACCATCCT
GGAACAGAACTGGAACGGTTACGCGAACAAAAAACCGACCAAACGTGTTGATAACT
ACTACGGTCTGACCCACTTCATCGAAATCCCGGTTAAAGCGCCGCCGGGTACCGTTGC
TCAGTCTGCTCCGAACCTGGCTGGTTCTCGTTCTTACCGTGAAACCGGTACCATGACC
GTTACCGTTGACGGTCTGAACGTTGCTCGTGCTCCGAACACCTCTGGTGAAATCGTTG
CTGTTTACAAACGTGGTGAATCTTTCGACTACGACACCGTTATCATCGACGTTAACGG
TTACGTTTGGGTTTCTTACATCGGTGGTTCTGGTAAACGTAACTACGTTGCTACCGGTG
CTACCAAAGACGGTAAACGTTTCGGTAACGCTTGGGGTACCTTCAAATAA
SEQ ID NO: 33
ATGGCGAAAACCCAGGCGGAAATCAACAAACGTCTGGATGCGTACGCGAAAG
GTACCGTTGATAGCCCGTACCGTATCAAAAAAGCGACCTCTTACGATCCGAGCTTCGG
TGTTATGGAAGCGGGTGCGATCGATGCGGATGGTTACGCTCACGCGCAGTGCCAGGA
TCTGATCACCGATTACGTTCTGTGGCTGACCGATAACAAAGTTCGTACCTGGGGTAAC
GCGAAAGATCAGATCAAACAGTCTTACGGTACCGGTTTCAAAATCCACGAAAACAAA
CCGTCTACCGTTCCGAAAAAAGGTTGGATCGCGGTTTTCACCAGCGGTTCTTACCAG
CAGTGGGGTCACATCGGTATCGTTTACGATGGTGGTAACACCAGCACCTTCACCATCC
TGGAACAGAACTGGAACGGTTACGCGAACAAAAAACCGACCAAACGTGTTGATAAC
TACTACGGTCTGACCCACTTCATCGAAATCCCGGTTAAAGCGCCGCCGGGTACCGTTG
CTCAGTCTGCTCCGAACCTGGCTGGTTCTCGTTCTTACCGTGAAACCGGTACCATGAC
CGTTACCGTTGACGCTCTGAACGTTCGTCGTGCTCCGAACACCTCTGGTGAAATCGTT
GCTGTTTACAAACGTGGTGAATCTTTCGACTACGACACCGTTATCATCGACGTTAACG
GTTACGTTTGGGTTTCTTACATCGGTGGTTCTGGTAAACGTAACTACGTTGCTACCGGT
GCTACCAAAGACGGTAAACGTTTCGGTAACGCTTGGGGTACCTTCAAATAA
SEQ ID NO: 34
ATGGCGAAAACCCAGGCGGAAATCAACAAACGTCTGGATGCGTACGCGAAAG
GTACCGTTGATAGCCCGTACCGTATCAAAAAAGCGACCTCTTACGATCCGGCTTTCGG
TGTTATGGAAGCGGGTGCGATCGCTGCGGATGGTTACTACCACGCGCAGTGCCAGGA
TCTGATCACCGATTACGTTCTGTGGCTGACCGATAACAAAGTTCGTACCTGGGGTAAC
GCGAAAGATCAGATCAAACAGTCTTACGGTACCGGTTTCAAAATCCACGAAAACAAA
CCGTCTACCGTTCCGAAAAAAGGTTGGATCGCGGTTTTCACCAGCGGTTCTTACCAG
CAGTGGGGTCACATCGGTATCGTTTACGATGGTGGTAACACCAGCACCTTCACCATCC
TGGAACAGAACTGGAACGGTTACGCGAACAAAAAACCGACCAAACGTGTTGATAAC
TACTACGGTCTGACCCACTTCATCGAAATCCCGGTTAAAGCGCCGCCGGGTACCGTTG
CTCAGTCTGCTCCGAACCTGGCTGGTTCTCGTTCTTACCGTGAAACCGGTACCATGAC
CGTTACCGTTGACGGTCTGAACGTTCGTCGTGCTCCGAACACCTCTGGTGAAATCGTT
GCTGTTTACAAACGTGGTGAATCTTTCGACTACGACACCGTTATCATCGACGTTAACG
GTTACGTTTGGGTTTCTTACATCGGTGGTTCTGGTAAACGTAACTACGTTGCTACCGGT
GCTACCAAAGACGGTAAACGTTTCGGTAACGCTTGGGGTACCTTCAAATAA
SEQ ID NO: 35
ATGGCGAAAACCCAGGCGGAAATCAACAAACGTCTGGATGCGTACGCGAAAG
GTACCGTTGATAGCCCGTACCGTATCAAAAAAGCGACCTCTTACGATCCGAGCTTCGG
TGTTATGGAAGCGGGTGCGATCGATGCGGATGGTTACTACCACGCGCAGTGCCAGGAT
CTGATCACCGATTACGTTCTGTGGCTGACCGATAACAAAGTTGCTACCTGGGGTAACG
CGAAAGATCAGATCAAACAGTCTTACGGTACCGGTTTCAAAATCCACGAAAACAAAC
CGTCTACCGTTCCGAAAAAAGGTTGGATCGCGGTTTTCACCAGCGGTTCTTACCAGC
AGTGGGGTCACATCGGTATCGTTTACGATGGTGGTAACACCAGCACCTTCACCATCCT
GGAACAGAACTGGAACGGTTACGCGAACAAAAAACCGACCAAACGTGTTGATAACT
ACTACGGTCTGACCCACTTCATCGAAATCCCGGTTAAAGCGCCGCCGGGTACCGTTGC
TCAGTCTGCTCCGAACCTGGCTGGTTCTCGTTCTTACCGTGAAACCGGTACCATGACC
GTTACCGTTGACGCTCTGAACGTTCGTCGTGCTCCGAACACCTCTGGTGAAATCGTTG
CTGTTTACAAACGTGGTGAATCTTTCGACTACGACACCGTTATCATCGACGTTAACGG
TTACGTTTGGGTTTCTTACATCGGTGGTTCTGGTAAACGTAACTACGTTGCTACCGGTG
CTACCAAAGACGGTAAACGTTTCGGTAACGCTTGGGGTACCTTCAAATAA
SEQ ID NO: 36
ATGGCGAAAACCCAGGCGGAAATCAACAAACGTCTGGATGCGTACGCGAAAG
GTACCGTTGATAGCCCGTACCGTATCAAAAAAGCGACCTCTTACGATCCGAGCTTCGG
TGTTATGGAAGCGGGTGCGATCGATGCGGATGGTTACTACCACGCGCAGTGCCAGGAT
CTGATCACCGATTACGTTCTGTGGCTGACCGATAACAAAGTTCGTACCTGGGGTAACG
CGAAAGATCAGATCAAACAGTCTTACGGTACCGGTTTCAAAATCCACGAAAACAAAC
CGTCTACCGTTCCGAAAAAAGGTTGGATCGCGGTTTTCACCAGCGGTTCTTACCAGC
AGTGGGGTCACATCGGTATCGTTTACGATGGTGGTAACACCAGCACCTTCACCATCCT
GGAACAGAACTGGAACGGTTACGCGAACAAAAAACCGACCAAACGTGTTGATAACT
ACTACGGTCTGACCCACTTCATCGAAATCCCGGTTAAAGCGCCGCCGGGTACCGTTGC
TCAGTCTGCTCCGAACCTGGCTGGTTCTCGTTCTTACCGTGAAACCGGTACCATGACC
GTTACCGTTGACGCTCTGAACGTTCGTCGTGCTCCGAACACCTCTGGTGAAATCGTTG
CTGTTTACAAACGTGGTGAATCTTTCGACTACGACACCGTTATCATCGACGTTAACGG
TTACGTTTGGGTTTCTTACATCGGTGGTTCTGGTAAACGTAACTACGTTGCTACCGGTG
CTACCAAAGACGGTAAACGTTTCGGTAACGCTTGGGGTACCTTCAAATAA
SEQ ID NO: 37
ATGGCGAAAACCCAGGCGGAAATCAACAAACGTCTGGATGCGTACGCGAAAG
GTACCGTTGATAGCCCGTACCGTATCAAAAAAGCGACCTCTTACGATCCGAGCTTCGG
TGTTATGGAAGCGGGTGCGATCGATGCGGATGGTTACGCTCACGCGCAGTGCCAGGA
TCTGATCACCGATTACGTTCTGTGGCTGACCGATAACAAAGTTGCTGCTTGGGGTAAC
GCGAAAGATCAGATCAAACAGTCTTACGGTACCGGTTTCAAAATCCACGAAAACAAA
CCGTCTACCGTTCCGAAAAAAGGTTGGATCGCGGTTTTCACCAGCGGTTCTTACCAG
CAGTGGGGTCACATCGGTATCGTTTACGATGGTGGTAACACCAGCACCTTCACCATCC
TGGAACAGAACTGGAACGGTTACGCGAACAAAAAACCGACCAAACGTGTTGATAAC
TACTACGGTCTGACCCACTTCATCGAAATCCCGGTTAAAGCGCCGCCGGGTACCGTTG
CTCAGTCTGCTCCGAACCTGGCTGGTTCTCGTTCTTACCGTGAAACCGGTACCATGAC
CGTTACCGTTGACGCTCTGAACGTTCGTCGTGCTCCGAACACCTCTGGTGAAATCGTT
GCTGTTTACAAACGTGGTGAATCTTTCGACTACGACACCGTTATCATCGACGTTAACG
GTTACGTTTGGGTTTCTTACATCGGTGGTTCTGGTAAACGTAACTACGTTGCTACCGGT
GCTACCAAAGACGGTAAACGTTTCGGTAACGCTTGGGGTACCTTCAAATAA
SEQ ID NO: 38
ATGGCGAAAACCCAGGCGGAAATCAACAAACGTCTGGATGCGTACGCGAAAG
GTACCGTTGATAGCCCGTACCGTATCAAAAAAGCGACCTCTTACGATCCGAGCTTCGG
TGTTATGGAAGCGGGTGCGATCGATGCGGATGGTTACTACCACGCGCAGTGCCAGGAT
CTGATCACCGATTACGTTCTGTGGCTGACCGATAACAAAGTTCGTGCTTGGGGTAACG
CGAAAGATCAGATCAAACAGTCTTACGGTACCGGTTTCAAAATCCACGAAAACAAAC
CGTCTACCGTTCCGAAAAAAGGTTGGATCGCGGTTTTCACCAGCGGTTCTTACCAGC
AGTGGGGTCACATCGGTATCGTTTACGATGGTGGTAACACCAGCACCTTCACCATCCT
GGAACAGAACTGGAACGGTTACGCGAACAAAAAACCGACCAAACGTGTTGATAACT
ACTACGGTCTGACCCACTTCATCGAAATCCCGGTTAAAGCGCCGCCGGGTACCGTTGC
TCAGTCTGCTCCGAACCTGGCTGGTTCTCGTTCTTACCGTGAAACCGGTACCATGACC
GTTACCGTTGACGCTCTGAACGTTCGTCGTGCTCCGAACACCTCTGGTGAAATCGTTG
CTGTTTACAAACGTGGTGAATCTTTCGACTACGACACCGTTATCATCGACGTTAACGG
TTACGTTTGGGTTTCTTACATCGGTGGTTCTGGTAAACGTAACTACGTTGCTACCGGTG
CTACCAAAGACGGTAAACGTTTCGGTAACGCTTGGGGTACCTTCAAATAA
SEQ ID NO: 39
ATGGCGAAAACCCAGGCGGAAATCAACAAACGTCTGGATGCGTACGCGAAAG
GTACCGTTGATAGCCCGTACCGTATCAAAAAAGCGACCTCTTACGATCCGGCTTTCGG
TGTTATGGAAGCGGGTGCGATCGATGCGGATGGTTACGCTCACGCGCAGTGCCAGGA
TCTGATCACCGATTACGTTCTGTGGCTGACCGATAACAAAGTTGCTACCTGGGGTAAC
GCGAAAGATCAGATCAAACAGTCTTACGGTACCGGTTTCAAAATCCACGAAAACAAA
CCGTCTACCGTTCCGAAAAAAGGTTGGATCGCGGTTTTCACCAGCGGTTCTTACCAG
CAGTGGGGTCACATCGGTATCGTTTACGATGGTGGTAACACCAGCACCTTCACCATCC
TGGAACAGAACTGGAACGGTTACGCGAACAAAAAACCGACCAAACGTGTTGATAAC
TACTACGGTCTGACCCACTTCATCGAAATCCCGGTTAAAGCGCCGCCGGGTACCGTTG
CTCAGTCTGCTCCGAACCTGGCTGGTTCTCGTTCTTACCGTGAAACCGGTACCATGAC
CGTTACCGTTGACGCTCTGAACGTTCGTCGTGCTCCGAACACCTCTGGTGAAATCGTT
GCTGTTTACAAACGTGGTGAATCTTTCGACTACGACACCGTTATCATCGACGTTAACG
GTTACGTTTGGGTTTCTTACATCGGTGGTTCTGGTAAACGTAACTACGTTGCTACCGGT
GCTACCAAAGACGGTAAACGTTTCGGTAACGCTTGGGGTACCTTCAAATAA
SEQ ID NO: 40
ATGGCGAAAACCCAGGCGGAAATCAACAAACGTCTGGATGCGTACGCGAAAG
GTACCGTTGATAGCCCGTACCGTATCAAAAAAGCGACCTCTTACGATCCGGCTTTCGG
TGTTATGGAAGCGGGTGCGATCGATGCGGATGGTTACGCTCACGCGCAGTGCCAGGA
TCTGATCACCGATTACGTTCTGTGGCTGACCGATAACAAAGTTCGTGCTTGGGGTAAC
GCGAAAGATCAGATCAAACAGTCTTACGGTACCGGTTTCAAAATCCACGAAAACAAA
CCGTCTACCGTTCCGAAAAAAGGTTGGATCGCGGTTTTCACCAGCGGTTCTTACCAG
CAGTGGGGTCACATCGGTATCGTTTACGATGGTGGTAACACCAGCACCTTCACCATCC
TGGAACAGAACTGGAACGGTTACGCGAACAAAAAACCGACCAAACGTGTTGATAAC
TACTACGGTCTGACCCACTTCATCGAAATCCCGGTTAAAGCGCCGCCGGGTACCGTTG
CTCAGTCTGCTCCGAACCTGGCTGGTTCTCGTTCTTACCGTGAAACCGGTACCATGAC
CGTTACCGTTGACGCTCTGAACGTTCGTCGTGCTCCGAACACCTCTGGTGAAATCGTT
GCTGTTTACAAACGTGGTGAATCTTTCGACTACGACACCGTTATCATCGACGTTAACG
GTTACGTTTGGGTTTCTTACATCGGTGGTTCTGGTAAACGTAACTACGTTGCTACCGGT
GCTACCAAAGACGGTAAACGTTTCGGTAACGCTTGGGGTACCTTCAAATAA
SEQ ID NO: 41
ATGGCGAAAACCCAGGCGGAAATCAACAAACGTCTGGATGCGTACGCGAAAG
GTACCGTTGATAGCCCGTACCGTATCAAAAAAGCGACCTCTTACGATCCGGCTTTCGG
TGTTATGGAAGCGGGTGCGATCGATGCGGATGGTTACTACCACGCGCAGTGCCAGGAT
CTGATCACCGATTACGTTCTGTGGCTGACCGATAACAAAGTTGCTGCTTGGGGTAACG
CGAAAGATCAGATCAAACAGTCTTACGGTACCGGTTTCAAAATCCACGAAAACAAAC
CGTCTACCGTTCCGAAAAAAGGTTGGATCGCGGTTTTCACCAGCGGTTCTTACCAGC
AGTGGGGTCACATCGGTATCGTTTACGATGGTGGTAACACCAGCACCTTCACCATCCT
GGAACAGAACTGGAACGGTTACGCGAACAAAAAACCGACCAAACGTGTTGATAACT
ACTACGGTCTGACCCACTTCATCGAAATCCCGGTTAAAGCGCCGCCGGGTACCGTTGC
TCAGTCTGCTCCGAACCTGGCTGGTTCTCGTTCTTACCGTGAAACCGGTACCATGACC
GTTACCGTTGACGCTCTGAACGTTCGTCGTGCTCCGAACACCTCTGGTGAAATCGTTG
CTGTTTACAAACGTGGTGAATCTTTCGACTACGACACCGTTATCATCGACGTTAACGG
TTACGTTTGGGTTTCTTACATCGGTGGTTCTGGTAAACGTAACTACGTTGCTACCGGTG
CTACCAAAGACGGTAAACGTTTCGGTAACGCTTGGGGTACCTTCAAATAA
SEQ ID NO: 42
ATGGCGAAAACCCAGGCGGAAATCAACAAACGTCTGGATGCGTACGCGAAAG
GTACCGTTGATAGCCCGTACCGTATCAAAAAAGCGACCTCTTACGATCCGGCTTTCGG
TGTTATGGAAGCGGGTGCGATCGATGCGGATGGTTACGCTCACGCGCAGTGCCAGGA
TCTGATCACCGATTACGTTCTGTGGCTGACCGATAACAAAGTTGCTGCTTGGGGTAAC
GCGAAAGATCAGATCAAACAGTCTTACGGTACCGGTTTCAAAATCCACGAAAACAAA
CCGTCTACCGTTCCGAAAAAAGGTTGGATCGCGGTTTTCACCAGCGGTTCTTACCAG
CAGTGGGGTCACATCGGTATCGTTTACGATGGTGGTAACACCAGCACCTTCACCATCC
TGGAACAGAACTGGAACGGTTACGCGAACAAAAAACCGACCAAACGTGTTGATAAC
TACTACGGTCTGACCCACTTCATCGAAATCCCGGTTAAAGCGCCGCCGGGTACCGTTG
CTCAGTCTGCTCCGAACCTGGCTGGTTCTCGTTCTTACCGTGAAACCGGTACCATGAC
CGTTACCGTTGACGCTCTGAACGTTCGTCGTGCTCCGAACACCTCTGGTGAAATCGTT
GCTGTTTACAAACGTGGTGAATCTTTCGACTACGACACCGTTATCATCGACGTTAACG
GTTACGTTTGGGTTTCTTACATCGGTGGTTCTGGTAAACGTAACTACGTTGCTACCGGT
GCTACCAAAGACGGTAAACGTTTCGGTAACGCTTGGGGTACCTTCAAATAA
SEQ ID NO: 43
ATGGCGAAAACCCAGGCGGAAATCAACAAACGTCTGGATGCGTACGCGAAAG
GTACCGTTGATAGCCCGTACCGTATCAAAAAAGCGACCTCTTACGATCCGGCTTTCGG
TGTTATGGAAGCGGGTGCGATCGATGCGGATGGTTACGCTCACGCGCAGTGCCAGGA
TCTGATCACCGATTACGTTCTGTGGCTGACCGATAACAAAGTTCGTACCTGGGGTAAC
GCGAAAGATCAGATCAAACAGTCTTACGGTACCGGTTTCAAAATCCACGAAAACAAA
CCGTCTACCGTTCCGAAAAAAGGTTGGATCGCGGTTTTCACCAGCGGTTCTTACCAG
CAGTGGGGTCACATCGGTATCGTTTACGATGGTGGTAACACCAGCACCTTCACCATCC
TGGAACAGAACTGGAACGGTTACGCGAACAAAAAACCGACCAAACGTGTTGATAAC
TACTACGGTCTGACCCACTTCATCGAAATCCCGGTTAAAGCGCCGCCGGGTACCGTTG
CTCAGTCTGCTCCGAACCTGGCTGGTTCTCGTTCTTACCGTGAAACCGGTACCATGAC
CGTTACCGTTGACGCTCTGAACGTTCGTCGTGCTCCGAACACCTCTGGTGAAATCGTT
GCTGTTTACAAACGTGGTGAATCTTTCGACTACGACACCGTTATCATCGACGTTAACG
GTTACGTTTGGGTTTCTTACATCGGTGGTTCTGGTAAACGTAACTACGTTGCTACCGGT
GCTACCAAAGACGGTAAACGTTTCGGTAACGCTTGGGGTACCTTCAAATAA
SEQ ID NO: 44
ATGGCGAAAACCCAGGCGGAAATCAACAAACGTCTGGATGCGTACGCGAAAG
GTACCGTTGATAGCCCGTACCGTATCAAAAAAGCGACCTCTTACGATCCGGCTTTCGG
TGTTATGGAAGCGGGTGCGATCGATGCGGATGGTTACTACCACGCGCAGTGCCAGGAT
CTGATCACCGATTACGTTCTGTGGCTGACCGATAACAAAGTTGCTACCTGGGGTAACG
CGAAAGATCAGATCAAACAGTCTTACGGTACCGGTTTCAAAATCCACGAAAACAAAC
CGTCTACCGTTCCGAAAAAAGGTTGGATCGCGGTTTTCACCAGCGGTTCTTACCAGC
AGTGGGGTCACATCGGTATCGTTTACGATGGTGGTAACACCAGCACCTTCACCATCCT
GGAACAGAACTGGAACGGTTACGCGAACAAAAAACCGACCAAACGTGTTGATAACT
ACTACGGTCTGACCCACTTCATCGAAATCCCGGTTAAAGCGCCGCCGGGTACCGTTGC
TCAGTCTGCTCCGAACCTGGCTGGTTCTCGTTCTTACCGTGAAACCGGTACCATGACC
GTTACCGTTGACGCTCTGAACGTTCGTCGTGCTCCGAACACCTCTGGTGAAATCGTTG
CTGTTTACAAACGTGGTGAATCTTTCGACTACGACACCGTTATCATCGACGTTAACGG
TTACGTTTGGGTTTCTTACATCGGTGGTTCTGGTAAACGTAACTACGTTGCTACCGGTG
CTACCAAAGACGGTAAACGTTTCGGTAACGCTTGGGGTACCTTCAAATAA
SEQ ID NO: 45
ATGGCGAAAACCCAGGCGGAAATCAACAAACGTCTGGATGCGTACGCGAAAG
GTACCGTTGATAGCCCGTACCGTATCAAAAAAGCGACCTCTTACGATCCGGCTTTCGG
TGTTATGGAAGCGGGTGCGATCGATGCGGATGGTTACTACCACGCGCAGTGCCAGGAT
CTGATCACCGATTACGTTCTGTGGCTGACCGATAACAAAGTTCGTGCTTGGGGTAACG
CGAAAGATCAGATCAAACAGTCTTACGGTACCGGTTTCAAAATCCACGAAAACAAAC
CGTCTACCGTTCCGAAAAAAGGTTGGATCGCGGTTTTCACCAGCGGTTCTTACCAGC
AGTGGGGTCACATCGGTATCGTTTACGATGGTGGTAACACCAGCACCTTCACCATCCT
GGAACAGAACTGGAACGGTTACGCGAACAAAAAACCGACCAAACGTGTTGATAACT
ACTACGGTCTGACCCACTTCATCGAAATCCCGGTTAAAGCGCCGCCGGGTACCGTTGC
TCAGTCTGCTCCGAACCTGGCTGGTTCTCGTTCTTACCGTGAAACCGGTACCATGACC
GTTACCGTTGACGCTCTGAACGTTCGTCGTGCTCCGAACACCTCTGGTGAAATCGTTG
CTGTTTACAAACGTGGTGAATCTTTCGACTACGACACCGTTATCATCGACGTTAACGG
TTACGTTTGGGTTTCTTACATCGGTGGTTCTGGTAAACGTAACTACGTTGCTACCGGTG
CTACCAAAGACGGTAAACGTTTCGGTAACGCTTGGGGTACCTTCAAATAA
SEQ ID NO: 46
ATGGCGAAAACCCAGGCGGAAATCAACAAACGTCTGGATGCGTACGCGAAAG
GTACCGTTGATAGCCCGTACCGTATCAAAAAAGCGACCTCTTACGATCCGAGCTTCGG
TGTTATGGAAGCGGGTGCGATCGATGCGGATGGTTACGCTCACGCGCAGTGCCAGGA
TCTGATCACCGATTACGTTCTGTGGCTGACCGATAACAAAGTTGCTACCTGGGGTAAC
GCGAAAGATCAGATCAAACAGTCTTACGGTACCGGTTTCAAAATCCACGAAAACAAA
CCGTCTACCGTTCCGAAAAAAGGTTGGATCGCGGTTTTCACCAGCGGTTCTTACCAG
CAGTGGGGTCACATCGGTATCGTTTACGATGGTGGTAACACCAGCACCTTCACCATCC
TGGAACAGAACTGGAACGGTTACGCGAACAAAAAACCGACCAAACGTGTTGATAAC
TACTACGGTCTGACCCACTTCATCGAAATCCCGGTTAAAGCGCCGCCGGGTACCGTTG
CTCAGTCTGCTCCGAACCTGGCTGGTTCTCGTTCTTACCGTGAAACCGGTACCATGAC
CGTTACCGTTGACGCTCTGAACGTTCGTCGTGCTCCGAACACCTCTGGTGAAATCGTT
GCTGTTTACAAACGTGGTGAATCTTTCGACTACGACACCGTTATCATCGACGTTAACG
GTTACGTTTGGGTTTCTTACATCGGTGGTTCTGGTAAACGTAACTACGTTGCTACCGGT
GCTACCAAAGACGGTAAACGTTTCGGTAACGCTTGGGGTACCTTCAAATAA
SEQ ID NO: 47
ATGGCGAAAACCCAGGCGGAAATCAACAAACGTCTGGATGCGTACGCGAAAG
GTACCGTTGATAGCCCGTACCGTATCAAAAAAGCGACCTCTTACGATCCGAGCTTCGG
TGTTATGGAAGCGGGTGCGATCGATGCGGATGGTTACGCTCACGCGCAGTGCCAGGA
TCTGATCACCGATTACGTTCTGTGGCTGACCGATAACAAAGTTCGTGCTTGGGGTAAC
GCGAAAGATCAGATCAAACAGTCTTACGGTACCGGTTTCAAAATCCACGAAAACAAA
CCGTCTACCGTTCCGAAAAAAGGTTGGATCGCGGTTTTCACCAGCGGTTCTTACCAG
CAGTGGGGTCACATCGGTATCGTTTACGATGGTGGTAACACCAGCACCTTCACCATCC
TGGAACAGAACTGGAACGGTTACGCGAACAAAAAACCGACCAAACGTGTTGATAAC
TACTACGGTCTGACCCACTTCATCGAAATCCCGGTTAAAGCGCCGCCGGGTACCGTTG
CTCAGTCTGCTCCGAACCTGGCTGGTTCTCGTTCTTACCGTGAAACCGGTACCATGAC
CGTTACCGTTGACGCTCTGAACGTTCGTCGTGCTCCGAACACCTCTGGTGAAATCGTT
GCTGTTTACAAACGTGGTGAATCTTTCGACTACGACACCGTTATCATCGACGTTAACG
GTTACGTTTGGGTTTCTTACATCGGTGGTTCTGGTAAACGTAACTACGTTGCTACCGGT
GCTACCAAAGACGGTAAACGTTTCGGTAACGCTTGGGGTACCTTCAAATAA
SEQ ID NO: 48
ATGGCGAAAACCCAGGCGGAAATCAACAAACGTCTGGATGCGTACGCGAAAG
GTACCGTTGATAGCCCGTACCGTATCAAAAAAGCGACCTCTTACGATCCGAGCTTCGG
TGTTATGGAAGCGGGTGCGATCGATGCGGATGGTTACTACCACGCGCAGTGCCAGGAT
CTGATCACCGATTACGTTCTGTGGCTGACCGATAACAAAGTTGCTGCTTGGGGTAACG
CGAAAGATCAGATCAAACAGTCTTACGGTACCGGTTTCAAAATCCACGAAAACAAAC
CGTCTACCGTTCCGAAAAAAGGTTGGATCGCGGTTTTCACCAGCGGTTCTTACCAGC
AGTGGGGTCACATCGGTATCGTTTACGATGGTGGTAACACCAGCACCTTCACCATCCT
GGAACAGAACTGGAACGGTTACGCGAACAAAAAACCGACCAAACGTGTTGATAACT
ACTACGGTCTGACCCACTTCATCGAAATCCCGGTTAAAGCGCCGCCGGGTACCGTTGC
TCAGTCTGCTCCGAACCTGGCTGGTTCTCGTTCTTACCGTGAAACCGGTACCATGACC
GTTACCGTTGACGCTCTGAACGTTCGTCGTGCTCCGAACACCTCTGGTGAAATCGTTG
CTGTTTACAAACGTGGTGAATCTTTCGACTACGACACCGTTATCATCGACGTTAACGG
TTACGTTTGGGTTTCTTACATCGGTGGTTCTGGTAAACGTAACTACGTTGCTACCGGTG
CTACCAAAGACGGTAAACGTTTCGGTAACGCTTGGGGTACCTTCAAATAA
SEQ ID NO: 49
MAKTQAEINKRLDAYAKGTVDSPYRX₁KKATSYDPX₂FGVMEAGAIX₃AX₄GYX₅
HAQCQX₆LITDYVLWLTDNKVX₇X₈WGNAKDQIKQSYGTGFKIHENKPSTVPKKGWIAV
FTSGSYX₉QWGHIGIVYDGGNTSTFTILEQNWNGYANKKPTKRVDNYYGLTHFIEIPVKA
PPGTVAQSAPNLAGSRSYRETGTMTVTVDX₁₀LNVX₁₁RAPNTSGEIVAVYKRGESFDYDT
VIIDVNGYVWVSYIGGSGKRNYVATGATKDGKRFGNAWGTFK

We claim:

1. A lysin composition, comprising
one or more lysins in combination,
wherein an amino acid sequence of the one or more lysins is selected from the group consisting of SEQ ID NO: 1 to SEQ ID NO: 24.

2. The lysin composition according to claim 1, wherein the amino acid sequence of the one or more lysins is selected from the group consisting of SEQ ID NO: 2, SEQ ID NO: 4, SEQ ID NO: 7, SEQ ID NO: 8, SEQ ID NO: 9, SEQ ID NO: 11, SEQ ID NO: 12, SEQ ID NO: 14, and SEQ ID NO: 22.

3. An expression vector encoding the lysin composition according to claim 1.

4. A recombinant strain comprising the expression vector according to claim 3.

5. A therapeutical composition comprising the lysin composition according to claim 1.

6. The therapeutical composition according to claim 5, comprising an immune serum prepared by using the lysin composition.

7. A pharmaceutical formulation comprising
the lysin composition according to claim 1, and
a pharmaceutically acceptable carrier.

8. A method for using the lysin composition according to claim 1, comprising
preparing a bacterial inhibitor, a lytic agent, or inactivator comprising the lysin composition of claim 1.

9. A method for using the lysin composition according to claim 1, comprising
preparing a medicament comprising the lysin composition of claim 1 for eradicating bacteria, preventing bacterial infections, or treating diseases caused by bacterial infections.

10. A method for using the lysin composition according to claim 1, comprising
applying the lysin composition of claim 1 in vitro for inhibiting or lysis of bacteria.

11. The method according to claim 8, wherein the bacteria belong to a genera Staphylococcus, Streptococcus, and/or Enterococcus.

12. The method according to claim 11, wherein the genera Staphylococcus is selected from Staphylococcus aureus, Staphylococcus epidermidis, Staphylococcus capitis, Staphylococcus pseudintermedius, Staphylococcus hominis, Staphylococcus haemolyticus, Staphylococcus saprophyticus, or Staphylococcus caprae;
the genera Streptococcus is selected from Streptococcus suis, Streptococcus dysgalactiae, Streptococcus agalactiae, Streptococcus pyogenes, Streptococcus mutans, or Streptococcus gallolyticus; and
the genera Enterococcus is selected from Enterococcus faecium or Enterococcus faecalis.

13. The method according to claim 9, wherein the bacteria belong to a genera Staphylococcus, Streptococcus, and/or Enterococcus.

14. The method according to claim 13, wherein the genera Staphylococcus is selected from Staphylococcus aureus, Staphylococcus epidermidis, Staphylococcus capitis, Staphylococcus pseudintermedius, Staphylococcus hominis, Staphylococcus haemolyticus, Staphylococcus saprophyticus, or Staphylococcus caprae;
the genera Streptococcus is selected from Streptococcus suis, Streptococcus dysgalactiae, Streptococcus agalactiae, Streptococcus pyogenes, Streptococcus mutans, or Streptococcus gallolyticus; and
the genera Enterococcus is selected from Enterococcus faecium or Enterococcus faecalis.

15. The method according to claim 10, wherein the bacteria belong to a genera Staphylococcus, Streptococcus, and/or Enterococcus.

16. The method according to claim 15, wherein the genera Staphylococcus is selected from Staphylococcus aureus, Staphylococcus epidermidis, Staphylococcus capitis, Staphylococcus pseudintermedius, Staphylococcus hominis, Staphylococcus haemolyticus, Staphylococcus saprophyticus, or Staphylococcus caprae;
the genera Streptococcus is selected from Streptococcus suis, Streptococcus dysgalactiae, Streptococcus agalactiae, Streptococcus pyogenes, Streptococcus mutans, or Streptococcus gallolyticus; and
the genera Enterococcus is selected from Enterococcus faecium or Enterococcus faecalis.


SEQUENCE LISTING (49 sequences)

SEQ ID NO: 1 - protein, 264 aa, synthetic construct
MAKTQAEINKRLDAYAKGTVDSPYRIKKATSYDPSFGVMEAGAIAAAGYYHAQCQALITDYVLWLTDNKVRTWGNAKDQIKQSYGTGFKIHENKPSTVPKKGWIAVFTSGSYQQWGHIGIVYDGGNTSTFTILEQNWNGYANKKPTKRVDNYYGLTHFIEIPVKAPPGTVAQSAPNLAGSRSYRETGTMTVTVDALNVRRAPNTSGEIVAVYKRGESFDYDTVIIDVNGYVWVSYIGGSGKRNYVATGATKDGKRFGNAWGTFK

SEQ ID NO: 2 - protein, 264 aa, synthetic construct
MAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYQQWGHIGIVYDGGNTSTFTILEQNWNGYANKKPTKRVDNYYGLTHFIEIPVKAPPGTVAQSAPNLAGSRSYRETGTMTVTVDALNVRRAPNTSGEIVAVYKRGESFDYDTVIIDVNGYVWVSYIGGSGKRNYVATGATKDGKRFGNAWGTFK

SEQ ID NO: 3 - protein, 264 aa, synthetic construct
MAKTQAEINKRLDAYAKGTVDSPYRIKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAPPGTVAQSAPNLAGSRSYRETGTMTVTVDALNVRRAPNTSGEIVAVYKRGESFDYDTVIIDVNGYVWVSYIGGSGKRNYVATGATKDGKRFGNAWGTFK

SEQ ID NO: 4 - protein, 264 aa, synthetic construct
MAKTQAEINKRLDAYAKGTVDSPYRIKKATSYDPAFGVMEAGAIDADGYYHAQCQDLITDYVLWLTDNKVRTWGNAKDQIKQSYGTGFKIHENKPSTVPKKGWIAVFTSGSYQQWGHIGIVYDGGNTSTFTILEQNWNGYANKKPTKRVDNYYGLTHFIEIPVKAPPGTVAQSAPNLAGSRSYRETGTMTVTVDALNVRRAPNTSGEIVAVYKRGESFDYDTVIIDVNGYVWVSYIGGSGKRNYVATGATKDGKRFGNAWGTFK

SEQ ID NO: 5 - protein, 264 aa, synthetic construct
MAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAPPGTVAQSAPNLAGSRSYRETGTMTVTVDALNVRRAPNTSGEIVAVYKRGESFDYDTVIIDVNGYVWVSYIGGSGKRNYVATGATKDGKRFGNAWGTFK

SEQ ID NO: 6 - protein, 264 aa, synthetic construct
MAKTQAEINKRLDAYAKGTVDSPYRIKKATSYDPAFGVMEAGAIAAAGYAHAQCQALITDYVLWLTDNKVAAWGNAKDQIKQSYGTGFKIHENKPSTVPKKGWIAVFTSGSYQQWGHIGIVYDGGNTSTFTILEQNWNGYANKKPTKRVDNYYGLTHFIEIPVKAPPGTVAQSAPNLAGSRSYRETGTMTVTVDGLNVARAPNTSGEIVAVYKRGESFDYDTVIIDVNGYVWVSYIGGSGKRNYVATGATKDGKRFGNAWGTFK

SEQ ID NO: 7 - protein, 264 aa, synthetic construct
MAKTQAEINKRLDAYAKGTVDSPYRIKKATSYDPSFGVMEAGAIDADGYYHAQCQDLITDYVLWLTDNKVRTWGNAKDQIKQSYGTGFKIHENKPSTVPKKGWIAVFTSGSYQQWGHIGIVYDGGNTSTFTILEQNWNGYANKKPTKRVDNYYGLTHFIEIPVKAPPGTVAQSAPNLAGSRSYRETGTMTVTVDALNVARAPNTSGEIVAVYKRGESFDYDTVIIDVNGYVWVSYIGGSGKRNYVATGATKDGKRFGNAWGTFK

SEQ ID NO: 8 - protein, 264 aa, synthetic construct
MAKTQAEINKRLDAYAKGTVDSPYRIKKATSYDPSFGVMEAGAIDADGYYHAQCQDLITDYVLWLTDNKVRTWGNAKDQIKQSYGTGFKIHENKPSTVPKKGWIAVFTSGSYQQWGHIGIVYDGGNTSTFTILEQNWNGYANKKPTKRVDNYYGLTHFIEIPVKAPPGTVAQSAPNLAGSRSYRETGTMTVTVDGLNVARAPNTSGEIVAVYKRGESFDYDTVIIDVNGYVWVSYIGGSGKRNYVATGATKDGKRFGNAWGTFK

SEQ ID NO: 9 - protein, 264 aa, synthetic construct
MAKTQAEINKRLDAYAKGTVDSPYRIKKATSYDPSFGVMEAGAIDADGYAHAQCQDLITDYVLWLTDNKVRTWGNAKDQIKQSYGTGFKIHENKPSTVPKKGWIAVFTSGSYQQWGHIGIVYDGGNTSTFTILEQNWNGYANKKPTKRVDNYYGLTHFIEIPVKAPPGTVAQSAPNLAGSRSYRETGTMTVTVDALNVRRAPNTSGEIVAVYKRGESFDYDTVIIDVNGYVWVSYIGGSGKRNYVATGATKDGKRFGNAWGTFK

SEQ ID NO: 10 - protein, 264 aa, synthetic construct
MAKTQAEINKRLDAYAKGTVDSPYRIKKATSYDPAFGVMEAGAIAADGYYHAQCQDLITDYVLWLTDNKVRTWGNAKDQIKQSYGTGFKIHENKPSTVPKKGWIAVFTSGSYQQWGHIGIVYDGGNTSTFTILEQNWNGYANKKPTKRVDNYYGLTHFIEIPVKAPPGTVAQSAPNLAGSRSYRETGTMTVTVDGLNVRRAPNTSGEIVAVYKRGESFDYDTVIIDVNGYVWVSYIGGSGKRNYVATGATKDGKRFGNAWGTFK

SEQ ID NO: 11 - protein, 264 aa, synthetic construct
MAKTQAEINKRLDAYAKGTVDSPYRIKKATSYDPSFGVMEAGAIDADGYYHAQCQDLITDYVLWLTDNKVATWGNAKDQIKQSYGTGFKIHENKPSTVPKKGWIAVFTSGSYQQWGHIGIVYDGGNTSTFTILEQNWNGYANKKPTKRVDNYYGLTHFIEIPVKAPPGTVAQSAPNLAGSRSYRETGTMTVTVDALNVRRAPNTSGEIVAVYKRGESFDYDTVIIDVNGYVWVSYIGGSGKRNYVATGATKDGKRFGNAWGTFK

SEQ ID NO: 12 - protein, 264 aa, synthetic construct
MAKTQAEINKRLDAYAKGTVDSPYRIKKATSYDPSFGVMEAGAIDADGYYHAQCQDLITDYVLWLTDNKVRTWGNAKDQIKQSYGTGFKIHENKPSTVPKKGWIAVFTSGSYQQWGHIGIVYDGGNTSTFTILEQNWNGYANKKPTKRVDNYYGLTHFIEIPVKAPPGTVAQSAPNLAGSRSYRETGTMTVTVDALNVRRAPNTSGEIVAVYKRGESFDYDTVIIDVNGYVWVSYIGGSGKRNYVATGATKDGKRFGNAWGTFK

SEQ ID NO: 13 - protein, 264 aa, synthetic construct
MAKTQAEINKRLDAYAKGTVDSPYRIKKATSYDPSFGVMEAGAIDADGYAHAQCQDLITDYVLWLTDNKVAAWGNAKDQIKQSYGTGFKIHENKPSTVPKKGWIAVFTSGSYQQWGHIGIVYDGGNTSTFTILEQNWNGYANKKPTKRVDNYYGLTHFIEIPVKAPPGTVAQSAPNLAGSRSYRETGTMTVTVDALNVRRAPNTSGEIVAVYKRGESFDYDTVIIDVNGYVWVSYIGGSGKRNYVATGATKDGKRFGNAWGTFK

SEQ ID NO: 14 - protein, 264 aa, synthetic construct
MAKTQAEINKRLDAYAKGTVDSPYRIKKATSYDPSFGVMEAGAIDADGYYHAQCQDLITDYVLWLTDNKVRAWGNAKDQIKQSYGTGFKIHENKPSTVPKKGWIAVFTSGSYQQWGHIGIVYDGGNTSTFTILEQNWNGYANKKPTKRVDNYYGLTHFIEIPVKAPPGTVAQSAPNLAGSRSYRETGTMTVTVDALNVRRAPNTSGEIVAVYKRGESFDYDTVIIDVNGYVWVSYIGGSGKRNYVATGATKDGKRFGNAWGTFK

SEQ ID NO: 15 - protein, 264 aa, synthetic construct
MAKTQAEINKRLDAYAKGTVDSPYRIKKATSYDPAFGVMEAGAIDADGYAHAQCQDLITDYVLWLTDNKVATWGNAKDQIKQSYGTGFKIHENKPSTVPKKGWIAVFTSGSYQQWGHIGIVYDGGNTSTFTILEQNWNGYANKKPTKRVDNYYGLTHFIEIPVKAPPGTVAQSAPNLAGSRSYRETGTMTVTVDALNVRRAPNTSGEIVAVYKRGESFDYDTVIIDVNGYVWVSYIGGSGKRNYVATGATKDGKRFGNAWGTFK

SEQ ID NO: 16 - protein, 264 aa, synthetic construct
MAKTQAEINKRLDAYAKGTVDSPYRIKKATSYDPAFGVMEAGAIDADGYAHAQCQDLITDYVLWLTDNKVRAWGNAKDQIKQSYGTGFKIHENKPSTVPKKGWIAVFTSGSYQQWGHIGIVYDGGNTSTFTILEQNWNGYANKKPTKRVDNYYGLTHFIEIPVKAPPGTVAQSAPNLAGSRSYRETGTMTVTVDALNVRRAPNTSGEIVAVYKRGESFDYDTVIIDVNGYVWVSYIGGSGKRNYVATGATKDGKRFGNAWGTFK

SEQ ID NO: 17 - protein, 264 aa, synthetic construct
MAKTQAEINKRLDAYAKGTVDSPYRIKKATSYDPAFGVMEAGAIDADGYYHAQCQDLITDYVLWLTDNKVAAWGNAKDQIKQSYGTGFKIHENKPSTVPKKGWIAVFTSGSYQQWGHIGIVYDGGNTSTFTILEQNWNGYANKKPTKRVDNYYGLTHFIEIPVKAPPGTVAQSAPNLAGSRSYRETGTMTVTVDALNVRRAPNTSGEIVAVYKRGESFDYDTVIIDVNGYVWVSYIGGSGKRNYVATGATKDGKRFGNAWGTFK

SEQ ID NO: 18 - protein, 264 aa, synthetic construct
MAKTQAEINKRLDAYAKGTVDSPYRIKKATSYDPAFGVMEAGAIDADGYAHAQCQDLITDYVLWLTDNKVAAWGNAKDQIKQSYGTGFKIHENKPSTVPKKGWIAVFTSGSYQQWGHIGIVYDGGNTSTFTILEQNWNGYANKKPTKRVDNYYGLTHFIEIPVKAPPGTVAQSAPNLAGSRSYRETGTMTVTVDALNVRRAPNTSGEIVAVYKRGESFDYDTVIIDVNGYVWVSYIGGSGKRNYVATGATKDGKRFGNAWGTFK

SEQ ID NO: 19 - protein, 264 aa, synthetic construct
MAKTQAEINKRLDAYAKGTVDSPYRIKKATSYDPAFGVMEAGAIDADGYAHAQCQDLITDYVLWLTDNKVRTWGNAKDQIKQSYGTGFKIHENKPSTVPKKGWIAVFTSGSYQQWGHIGIVYDGGNTSTFTILEQNWNGYANKKPTKRVDNYYGLTHFIEIPVKAPPGTVAQSAPNLAGSRSYRETGTMTVTVDALNVRRAPNTSGEIVAVYKRGESFDYDTVIIDVNGYVWVSYIGGSGKRNYVATGATKDGKRFGNAWGTFK

SEQ ID NO: 20 - protein, 264 aa, synthetic construct
MAKTQAEINKRLDAYAKGTVDSPYRIKKATSYDPAFGVMEAGAIDADGYYHAQCQDLITDYVLWLTDNKVATWGNAKDQIKQSYGTGFKIHENKPSTVPKKGWIAVFTSGSYQQWGHIGIVYDGGNTSTFTILEQNWNGYANKKPTKRVDNYYGLTHFIEIPVKAPPGTVAQSAPNLAGSRSYRETGTMTVTVDALNVRRAPNTSGEIVAVYKRGESFDYDTVIIDVNGYVWVSYIGGSGKRNYVATGATKDGKRFGNAWGTFK

SEQ ID NO: 21 - protein, 264 aa, synthetic construct
MAKTQAEINKRLDAYAKGTVDSPYRIKKATSYDPAFGVMEAGAIDADGYYHAQCQDLITDYVLWLTDNKVRAWGNAKDQIKQSYGTGFKIHENKPSTVPKKGWIAVFTSGSYQQWGHIGIVYDGGNTSTFTILEQNWNGYANKKPTKRVDNYYGLTHFIEIPVKAPPGTVAQSAPNLAGSRSYRETGTMTVTVDALNVRRAPNTSGEIVAVYKRGESFDYDTVIIDVNGYVWVSYIGGSGKRNYVATGATKDGKRFGNAWGTFK

SEQ ID NO: 22 - protein, 264 aa, synthetic construct
MAKTQAEINKRLDAYAKGTVDSPYRIKKATSYDPSFGVMEAGAIDADGYAHAQCQDLITDYVLWLTDNKVATWGNAKDQIKQSYGTGFKIHENKPSTVPKKGWIAVFTSGSYQQWGHIGIVYDGGNTSTFTILEQNWNGYANKKPTKRVDNYYGLTHFIEIPVKAPPGTVAQSAPNLAGSRSYRETGTMTVTVDALNVRRAPNTSGEIVAVYKRGESFDYDTVIIDVNGYVWVSYIGGSGKRNYVATGATKDGKRFGNAWGTFK

SEQ ID NO: 23 - protein, 264 aa, synthetic construct
MAKTQAEINKRLDAYAKGTVDSPYRIKKATSYDPSFGVMEAGAIDADGYAHAQCQDLITDYVLWLTDNKVRAWGNAKDQIKQSYGTGFKIHENKPSTVPKKGWIAVFTSGSYQQWGHIGIVYDGGNTSTFTILEQNWNGYANKKPTKRVDNYYGLTHFIEIPVKAPPGTVAQSAPNLAGSRSYRETGTMTVTVDALNVRRAPNTSGEIVAVYKRGESFDYDTVIIDVNGYVWVSYIGGSGKRNYVATGATKDGKRFGNAWGTFK

SEQ ID NO: 24 - protein, 264 aa, synthetic construct
MAKTQAEINKRLDAYAKGTVDSPYRIKKATSYDPSFGVMEAGAIDADGYYHAQCQDLITDYVLWLTDNKVAAWGNAKDQIKQSYGTGFKIHENKPSTVPKKGWIAVFTSGSYQQWGHIGIVYDGGNTSTFTILEQNWNGYANKKPTKRVDNYYGLTHFIEIPVKAPPGTVAQSAPNLAGSRSYRETGTMTVTVDALNVRRAPNTSGEIVAVYKRGESFDYDTVIIDVNGYVWVSYIGGSGKRNYVATGATKDGKRFGNAWGTFK

SEQ ID NO: 25 - DNA, 795 bp, synthetic construct
  source 1..795: mol_type = other DNA
atggcgaaaacccaggcggaaatcaacaaacgtctggatgcgtacgcgaaaggtaccgttgatagcccgtaccgtatcaaaaaagcgacctcttacgatccgagcttcggtgttatggaagcgggtgcgatcgctgcggctggttactaccacgcgcagtgccaggctctgatcaccgattacgttctgtggctgaccgataacaaagttcgtacctggggtaacgcgaaagatcagatcaaacagtcttacggtaccggtttcaaaatccacgaaaacaaaccgtctaccgttccgaaaaaaggttggatcgcggttttcaccagcggttcttaccagcagtggggtcacatcggtatcgtttacgatggtggtaacaccagcaccttcaccatcctggaacagaactggaacggttacgcgaacaaaaaaccgaccaaacgtgttgataactactacggtctgacccacttcatcgaaatcccggttaaagcgccgccgggtaccgttgctcagtctgctccgaacctggctggttctcgttcttaccgtgaaaccggtaccatgaccgttaccgttgacgctctgaacgttcgtcgtgctccgaacacctctggtgaaatcgttgctgtttacaaacgtggtgaatctttcgactacgacaccgttatcatcgacgttaacggttacgtttgggtttcttacatcggtggttctggtaaacgtaactacgttgctaccggtgctaccaaagacggtaaacgtttcggtaacgcttggggtaccttcaaataa

SEQ ID NO: 26 - DNA, 795 bp, synthetic construct
  source 1..795: mol_type = other DNA
atggcgaaaacccaggcggaaatcaacaaacgtctggatgcgtacgcgaaaggtaccgttgatagcccgtaccgtgtcaaaaaagcgacctcttacgatccgagcttcggtgttatggaagcgggtgcgatcgatgcggatggttactaccacgcgcagtgccaggatctgatcaccgattacgttctgtggctgaccgataacaaagttcgtacctggggtaacgcgaaagatcagatcaaacagtcttacggtaccggtttcaaaatccacgaaaacaaaccgtctaccgttccgaaaaaaggttggatcgcggttttcaccagcggttcttaccagcagtggggtcacatcggtatcgtttacgatggtggtaacaccagcaccttcaccatcctggaacagaactggaacggttacgcgaacaaaaaaccgaccaaacgtgttgataactactacggtctgacccacttcatcgaaatcccggttaaagcgccgccgggtaccgttgctcagtctgctccgaacctggctggttctcgttcttaccgtgaaaccggtaccatgaccgttaccgttgacgctctgaacgttcgtcgtgctccgaacacctctggtgaaatcgttgctgtttacaaacgtggtgaatctttcgactacgacaccgttatcatcgacgttaacggttacgtttgggtttcttacatcggtggttctggtaaacgtaactacgttgctaccggtgctaccaaagacggtaaacgtttcggtaacgcttggggtaccttcaaataa

SEQ ID NO: 27 - DNA, 795 bp, synthetic construct
  source 1..795: mol_type = other DNA
atggcgaaaacccaggcggaaatcaacaaacgtctggatgcgtacgcgaaaggtaccgttgatagcccgtaccgtatcaaaaaagcgacctcttacgatccgagcttcggtgttatggaagcgggtgcgatcgatgcggatggttactaccacgcgcagtgccaggatctgatcaccgattacgttctgtggctgaccgataacaaagttcgtacctggggtaacgcgaaagatcagatcaaacagtcttacggtaccggtttcaaaatccacgaaaacaaaccgtctaccgttccgaaaaaaggttggatcgcggttttcaccagcggttcttacgaacagtggggtcacatcggtatcgtttacgatggtggtaacaccagcaccttcaccatcctggaacagaactggaacggttacgcgaacaaaaaaccgaccaaacgtgttgataactactacggtctgacccacttcatcgaaatcccggttaaagcgccgccgggtaccgttgctcagtctgctccgaacctggctggttctcgttcttaccgtgaaaccggtaccatgaccgttaccgttgacgctctgaacgttcgtcgtgctccgaacacctctggtgaaatcgttgctgtttacaaacgtggtgaatctttcgactacgacaccgttatcatcgacgttaacggttacgtttgggtttcttacatcggtggttctggtaaacgtaactacgttgctaccggtgctaccaaagacggtaaacgtttcggtaacgcttggggtaccttcaaataa

SEQ ID NO: 28 - DNA, 795 bp, synthetic construct
  source 1..795: mol_type = other DNA
atggcgaaaacccaggcggaaatcaacaaacgtctggatgcgtacgcgaaaggtaccgttgatagcccgtaccgtatcaaaaaagcgacctcttacgatccggctttcggtgttatggaagcgggtgcgatcgatgcggatggttactaccacgcgcagtgccaggatctgatcaccgattacgttctgtggctgaccgataacaaagttcgtacctggggtaacgcgaaagatcagatcaaacagtcttacggtaccggtttcaaaatccacgaaaacaaaccgtctaccgttccgaaaaaaggttggatcgcggttttcaccagcggttcttaccagcagtggggtcacatcggtatcgtttacgatggtggtaacaccagcaccttcaccatcctggaacagaactggaacggttacgcgaacaaaaaaccgaccaaacgtgttgataactactacggtctgacccacttcatcgaaatcccggttaaagcgccgccgggtaccgttgctcagtctgctccgaacctggctggttctcgttcttaccgtgaaaccggtaccatgaccgttaccgttgacgctctgaacgttcgtcgtgctccgaacacctctggtgaaatcgttgctgtttacaaacgtggtgaatctttcgactacgacaccgttatcatcgacgttaacggttacgtttgggtttcttacatcggtggttctggtaaacgtaactacgttgctaccggtgctaccaaagacggtaaacgtttcggtaacgcttggggtaccttcaaataa

SEQ ID NO: 29 - DNA, 795 bp, synthetic construct
  source 1..795: mol_type = other DNA
atggcgaaaacccaggcggaaatcaacaaacgtctggatgcgtacgcgaaaggtaccgttgatagcccgtaccgtgtcaaaaaagcgacctcttacgatccgagcttcggtgttatggaagcgggtgcgatcgatgcggatggttactaccacgcgcagtgccaggatctgatcaccgattacgttctgtggctgaccgataacaaagttcgtacctggggtaacgcgaaagatcagatcaaacagtcttacggtaccggtttcaaaatccacgaaaacaaaccgtctaccgttccgaaaaaaggttggatcgcggttttcaccagcggttcttacgaacagtggggtcacatcggtatcgtttacgatggtggtaacaccagcaccttcaccatcctggaacagaactggaacggttacgcgaacaaaaaaccgaccaaacgtgttgataactactacggtctgacccacttcatcgaaatcccggttaaagcgccgccgggtaccgttgctcagtctgctccgaacctggctggttctcgttcttaccgtgaaaccggtaccatgaccgttaccgttgacgctctgaacgttcgtcgtgctccgaacacctctggtgaaatcgttgctgtttacaaacgtggtgaatctttcgactacgacaccgttatcatcgacgttaacggttacgtttgggtttcttacatcggtggttctggtaaacgtaactacgttgctaccggtgctaccaaagacggtaaacgtttcggtaacgcttggggtaccttcaaataa

SEQ ID NO: 30 - DNA, 795 bp, synthetic construct
  source 1..795: mol_type = other DNA
atggcgaaaacccaggcggaaatcaacaaacgtctggatgcgtacgcgaaaggtaccgttgatagcccgtaccgtatcaaaaaagcgacctcttacgatccggctttcggtgttatggaagcgggtgcgatcgctgcggctggttacgctcacgcgcagtgccaggctctgatcaccgattacgttctgtggctgaccgataacaaagttgctgcttggggtaacgcgaaagatcagatcaaacagtcttacggtaccggtttcaaaatccacgaaaacaaaccgtctaccgttccgaaaaaaggttggatcgcggttttcaccagcggttcttaccagcagtggggtcacatcggtatcgtttacgatggtggtaacaccagcaccttcaccatcctggaacagaactggaacggttacgcgaacaaaaaaccgaccaaacgtgttgataactactacggtctgacccacttcatcgaaatcccggttaaagcgccgccgggtaccgttgctcagtctgctccgaacctggctggttctcgttcttaccgtgaaaccggtaccatgaccgttaccgttgacggtctgaacgttgctcgtgctccgaacacctctggtgaaatcgttgctgtttacaaacgtggtgaatctttcgactacgacaccgttatcatcgacgttaacggttacgtttgggtttcttacatcggtggttctggtaaacgtaactacgttgctaccggtgctaccaaagacggtaaacgtttcggtaacgcttggggtaccttcaaataa

SEQ ID NO: 31 - DNA, 795 bp, synthetic construct
  source 1..795: mol_type = other DNA
atggcgaaaacccaggcggaaatcaacaaacgtctggatgcgtacgcgaaaggtaccgttgatagcccgtaccgtatcaaaaaagcgacctcttacgatccgagcttcggtgttatggaagcgggtgcgatcgatgcggatggttactaccacgcgcagtgccaggatctgatcaccgattacgttctgtggctgaccgataacaaagttcgtacctggggtaacgcgaaagatcagatcaaacagtcttacggtaccggtttcaaaatccacgaaaacaaaccgtctaccgttccgaaaaaaggttggatcgcggttttcaccagcggttcttaccagcagtggggtcacatcggtatcgtttacgatggtggtaacaccagcaccttcaccatcctggaacagaactggaacggttacgcgaacaaaaaaccgaccaaacgtgttgataactactacggtctgacccacttcatcgaaatcccggttaaagcgccgccgggtaccgttgctcagtctgctccgaacctggctggttctcgttcttaccgtgaaaccggtaccatgaccgttaccgttgacgctctgaacgttgctcgtgctccgaacacctctggtgaaatcgttgctgtttacaaacgtggtgaatctttcgactacgacaccgttatcatcgacgttaacggttacgtttgggtttcttacatcggtggttctggtaaacgtaactacgttgctaccggtgctaccaaagacggtaaacgtttcggtaacgcttggggtaccttcaaataa

SEQ ID NO: 32 - DNA, 795 bp, synthetic construct
  source 1..795: mol_type = other DNA
atggcgaaaacccaggcggaaatcaacaaacgtctggatgcgtacgcgaaaggtaccgttgatagcccgtaccgtatcaaaaaagcgacctcttacgatccgagcttcggtgttatggaagcgggtgcgatcgatgcggatggttactaccacgcgcagtgccaggatctgatcaccgattacgttctgtggctgaccgataacaaagttcgtacctggggtaacgcgaaagatcagatcaaacagtcttacggtaccggtttcaaaatccacgaaaacaaaccgtctaccgttccgaaaaaaggttggatcgcggttttcaccagcggttcttaccagcagtggggtcacatcggtatcgtttacgatggtggtaacaccagcaccttcaccatcctggaacagaactggaacggttacgcgaacaaaaaaccgaccaaacgtgttgataactactacggtctgacccacttcatcgaaatcccggttaaagcgccgccgggtaccgttgctcagtctgctccgaacctggctggttctcgttcttaccgtgaaaccggtaccatgaccgttaccgttgacggtctgaacgttgctcgtgctccgaacacctctggtgaaatcgttgctgtttacaaacgtggtgaatctttcgactacgacaccgttatcatcgacgttaacggttacgtttgggtttcttacatcggtggttctggtaaacgtaactacgttgctaccggtgctaccaaagacggtaaacgtttcggtaacgcttggggtaccttcaaataa

SEQ ID NO: 33 - DNA, 795 bp, synthetic construct
  source 1..795: mol_type = other DNA
atggcgaaaacccaggcggaaatcaacaaacgtctggatgcgtacgcgaaaggtaccgttgatagcccgtaccgtatcaaaaaagcgacctcttacgatccgagcttcggtgttatggaagcgggtgcgatcgatgcggatggttacgctcacgcgcagtgccaggatctgatcaccgattacgttctgtggctgaccgataacaaagttcgtacctggggtaacgcgaaagatcagatcaaacagtcttacggtaccggtttcaaaatccacgaaaacaaaccgtctaccgttccgaaaaaaggttggatcgcggttttcaccagcggttcttaccagcagtggggtcacatcggtatcgtttacgatggtggtaacaccagcaccttcaccatcctggaacagaactggaacggttacgcgaacaaaaaaccgaccaaacgtgttgataactactacggtctgacccacttcatcgaaatcccggttaaagcgccgccgggtaccgttgctcagtctgctccgaacctggctggttctcgttcttaccgtgaaaccggtaccatgaccgttaccgttgacgctctgaacgttcgtcgtgctccgaacacctctggtgaaatcgttgctgtttacaaacgtggtgaatctttcgactacgacaccgttatcatcgacgttaacggttacgtttgggtttcttacatcggtggttctggtaaacgtaactacgttgctaccggtgctaccaaagacggtaaacgtttcggtaacgcttggggtaccttcaaataa

SEQ ID NO: 34 - DNA, 795 bp, synthetic construct
  source 1..795: mol_type = other DNA
atggcgaaaacccaggcggaaatcaacaaacgtctggatgcgtacgcgaaaggtaccgttgatagcccgtaccgtatcaaaaaagcgacctcttacgatccggctttcggtgttatggaagcgggtgcgatcgctgcggatggttactaccacgcgcagtgccaggatctgatcaccgattacgttctgtggctgaccgataacaaagttcgtacctggggtaacgcgaaagatcagatcaaacagtcttacggtaccggtttcaaaatccacgaaaacaaaccgtctaccgttccgaaaaaaggttggatcgcggttttcaccagcggttcttaccagcagtggggtcacatcggtatcgtttacgatggtggtaacaccagcaccttcaccatcctggaacagaactggaacggttacgcgaacaaaaaaccgaccaaacgtgttgataactactacggtctgacccacttcatcgaaatcccggttaaagcgccgccgggtaccgttgctcagtctgctccgaacctggctggttctcgttcttaccgtgaaaccggtaccatgaccgttaccgttgacggtctgaacgttcgtcgtgctccgaacacctctggtgaaatcgttgctgtttacaaacgtggtgaatctttcgactacgacaccgttatcatcgacgttaacggttacgtttgggtttcttacatcggtggttctggtaaacgtaactacgttgctaccggtgctaccaaagacggtaaacgtttcggtaacgcttggggtaccttcaaataa

SEQ ID NO: 35 - DNA, 795 bp, synthetic construct
  source 1..795: mol_type = other DNA
atggcgaaaacccaggcggaaatcaacaaacgtctggatgcgtacgcgaaaggtaccgttgatagcccgtaccgtatcaaaaaagcgacctcttacgatccgagcttcggtgttatggaagcgggtgcgatcgatgcggatggttactaccacgcgcagtgccaggatctgatcaccgattacgttctgtggctgaccgataacaaagttgctacctggggtaacgcgaaagatcagatcaaacagtcttacggtaccggtttcaaaatccacgaaaacaaaccgtctaccgttccgaaaaaaggttggatcgcggttttcaccagcggttcttaccagcagtggggtcacatcggtatcgtttacgatggtggtaacaccagcaccttcaccatcctggaacagaactggaacggttacgcgaacaaaaaaccgaccaaacgtgttgataactactacggtctgacccacttcatcgaaatcccggttaaagcgccgccgggtaccgttgctcagtctgctccgaacctggctggttctcgttcttaccgtgaaaccggtaccatgaccgttaccgttgacgctctgaacgttcgtcgtgctccgaacacctctggtgaaatcgttgctgtttacaaacgtggtgaatctttcgactacgacaccgttatcatcgacgttaacggttacgtttgggtttcttacatcggtggttctggtaaacgtaactacgttgctaccggtgctaccaaagacggtaaacgtttcggtaacgcttggggtaccttcaaataa

SEQ ID NO: 36 - DNA, 795 bp, synthetic construct
  source 1..795: mol_type = other DNA
atggcgaaaacccaggcggaaatcaacaaacgtctggatgcgtacgcgaaaggtaccgttgatagcccgtaccgtatcaaaaaagcgacctcttacgatccgagcttcggtgttatggaagcgggtgcgatcgatgcggatggttactaccacgcgcagtgccaggatctgatcaccgattacgttctgtggctgaccgataacaaagttcgtacctggggtaacgcgaaagatcagatcaaacagtcttacggtaccggtttcaaaatccacgaaaacaaaccgtctaccgttccgaaaaaaggttggatcgcggttttcaccagcggttcttaccagcagtggggtcacatcggtatcgtttacgatggtggtaacaccagcaccttcaccatcctggaacagaactggaacggttacgcgaacaaaaaaccgaccaaacgtgttgataactactacggtctgacccacttcatcgaaatcccggttaaagcgccgccgggtaccgttgctcagtctgctccgaacctggctggttctcgttcttaccgtgaaaccggtaccatgaccgttaccgttgacgctctgaacgttcgtcgtgctccgaacacctctggtgaaatcgttgctgtttacaaacgtggtgaatctttcgactacgacaccgttatcatcgacgttaacggttacgtttgggtttcttacatcggtggttctggtaaacgtaactacgttgctaccggtgctaccaaagacggtaaacgtttcggtaacgcttggggtaccttcaaataa

SEQ ID NO: 37 - DNA, 795 bp, synthetic construct
  source 1..795: mol_type = other DNA
atggcgaaaacccaggcggaaatcaacaaacgtctggatgcgtacgcgaaaggtaccgttgatagcccgtaccgtatcaaaaaagcgacctcttacgatccgagcttcggtgttatggaagcgggtgcgatcgatgcggatggttacgctcacgcgcagtgccaggatctgatcaccgattacgttctgtggctgaccgataacaaagttgctgcttggggtaacgcgaaagatcagatcaaacagtcttacggtaccggtttcaaaatccacgaaaacaaaccgtctaccgttccgaaaaaaggttggatcgcggttttcaccagcggttcttaccagcagtggggtcacatcggtatcgtttacgatggtggtaacaccagcaccttcaccatcctggaacagaactggaacggttacgcgaacaaaaaaccgaccaaacgtgttgataactactacggtctgacccacttcatcgaaatcccggttaaagcgccgccgggtaccgttgctcagtctgctccgaacctggctggttctcgttcttaccgtgaaaccggtaccatgaccgttaccgttgacgctctgaacgttcgtcgtgctccgaacacctctggtgaaatcgttgctgtttacaaacgtggtgaatctttcgactacgacaccgttatcatcgacgttaacggttacgtttgggtttcttacatcggtggttctggtaaacgtaactacgttgctaccggtgctaccaaagacggtaaacgtttcggtaacgcttggggtaccttcaaataa

SEQ ID NO: 38 - DNA, 795 bp, synthetic construct
  source 1..795: mol_type = other DNA
atggcgaaaacccaggcggaaatcaacaaacgtctggatgcgtacgcgaaaggtaccgttgatagcccgtaccgtatcaaaaaagcgacctcttacgatccgagcttcggtgttatggaagcgggtgcgatcgatgcggatggttactaccacgcgcagtgccaggatctgatcaccgattacgttctgtggctgaccgataacaaagttcgtgcttggggtaacgcgaaagatcagatcaaacagtcttacggtaccggtttcaaaatccacgaaaacaaaccgtctaccgttccgaaaaaaggttggatcgcggttttcaccagcggttcttaccagcagtggggtcacatcggtatcgtttacgatggtggtaacaccagcaccttcaccatcctggaacagaactggaacggttacgcgaacaaaaaaccgaccaaacgtgttgataactactacggtctgacccacttcatcgaaatcccggttaaagcgccgccgggtaccgttgctcagtctgctccgaacctggctggttctcgttcttaccgtgaaaccggtaccatgaccgttaccgttgacgctctgaacgttcgtcgtgctccgaacacctctggtgaaatcgttgctgtttacaaacgtggtgaatctttcgactacgacaccgttatcatcgacgttaacggttacgtttgggtttcttacatcggtggttctggtaaacgtaactacgttgctaccggtgctaccaaagacggtaaacgtttcggtaacgcttggggtaccttcaaataa

SEQ ID NO: 39 - DNA, 795 bp, synthetic construct
  source 1..795: mol_type = other DNA
atggcgaaaacccaggcggaaatcaacaaacgtctggatgcgtacgcgaaaggtaccgttgatagcccgtaccgtatcaaaaaagcgacctcttacgatccggctttcggtgttatggaagcgggtgcgatcgatgcggatggttacgctcacgcgcagtgccaggatctgatcaccgattacgttctgtggctgaccgataacaaagttgctacctggggtaacgcgaaagatcagatcaaacagtcttacggtaccggtttcaaaatccacgaaaacaaaccgtctaccgttccgaaaaaaggttggatcgcggttttcaccagcggttcttaccagcagtggggtcacatcggtatcgtttacgatggtggtaacaccagcaccttcaccatcctggaacagaactggaacggttacgcgaacaaaaaaccgaccaaacgtgttgataactactacggtctgacccacttcatcgaaatcccggttaaagcgccgccgggtaccgttgctcagtctgctccgaacctggctggttctcgttcttaccgtgaaaccggtaccatgaccgttaccgttgacgctctgaacgttcgtcgtgctccgaacacctctggtgaaatcgttgctgtttacaaacgtggtgaatctttcgactacgacaccgttatcatcgacgttaacggttacgtttgggtttcttacatcggtggttctggtaaacgtaactacgttgctaccggtgctaccaaagacggtaaacgtttcggtaacgcttggggtaccttcaaataa

SEQ ID NO: 40 - DNA, 795 bp, synthetic construct
  source 1..795: mol_type = other DNA
atggcgaaaacccaggcggaaatcaacaaacgtctggatgcgtacgcgaaaggtaccgttgatagcccgtaccgtatcaaaaaagcgacctcttacgatccggctttcggtgttatggaagcgggtgcgatcgatgcggatggttacgctcacgcgcagtgccaggatctgatcaccgattacgttctgtggctgaccgataacaaagttcgtgcttggggtaacgcgaaagatcagatcaaacagtcttacggtaccggtttcaaaatccacgaaaacaaaccgtctaccgttccgaaaaaaggttggatcgcggttttcaccagcggttcttaccagcagtggggtcacatcggtatcgtttacgatggtggtaacaccagcaccttcaccatcctggaacagaactggaacggttacgcgaacaaaaaaccgaccaaacgtgttgataactactacggtctgacccacttcatcgaaatcccggttaaagcgccgccgggtaccgttgctcagtctgctccgaacctggctggttctcgttcttaccgtgaaaccggtaccatgaccgttaccgttgacgctctgaacgttcgtcgtgctccgaacacctctggtgaaatcgttgctgtttacaaacgtggtgaatctttcgactacgacaccgttatcatcgacgttaacggttacgtttgggtttcttacatcggtggttctggtaaacgtaactacgttgctaccggtgctaccaaagacggtaaacgtttcggtaacgcttggggtaccttcaaataa

SEQ ID NO: 41 - DNA, 795 bp, synthetic construct
  source 1..795: mol_type = other DNA
atggcgaaaacccaggcggaaatcaacaaacgtctggatgcgtacgcgaaaggtaccgttgatagcccgtaccgtatcaaaaaagcgacctcttacgatccggctttcggtgttatggaagcgggtgcgatcgatgcggatggttactaccacgcgcagtgccaggatctgatcaccgattacgttctgtggctgaccgataacaaagttgctgcttggggtaacgcgaaagatcagatcaaacagtcttacggtaccggtttcaaaatccacgaaaacaaaccgtctaccgttccgaaaaaaggttggatcgcggttttcaccagcggttcttaccagcagtggggtcacatcggtatcgtttacgatggtggtaacaccagcaccttcaccatcctggaacagaactggaacggttacgcgaacaaaaaaccgaccaaacgtgttgataactactacggtctgacccacttcatcgaaatcccggttaaagcgccgccgggtaccgttgctcagtctgctccgaacctggctggttctcgttcttaccgtgaaaccggtaccatgaccgttaccgttgacgctctgaacgttcgtcgtgctccgaacacctctggtgaaatcgttgctgtttacaaacgtggtgaatctttcgactacgacaccgttatcatcgacgttaacggttacgtttgggtttcttacatcggtggttctggtaaacgtaactacgttgctaccggtgctaccaaagacggtaaacgtttcggtaacgcttggggtaccttcaaataa

SEQ ID NO: 42 - DNA, 795 bp, synthetic construct
  source 1..795: mol_type = other DNA
atggcgaaaacccaggcggaaatcaacaaacgtctggatgcgtacgcgaaaggtaccgttgatagcccgtaccgtatcaaaaaagcgacctcttacgatccggctttcggtgttatggaagcgggtgcgatcgatgcggatggttacgctcacgcgcagtgccaggatctgatcaccgattacgttctgtggctgaccgataacaaagttgctgcttggggtaacgcgaaagatcagatcaaacagtcttacggtaccggtttcaaaatccacgaaaacaaaccgtctaccgttccgaaaaaaggttggatcgcggttttcaccagcggttcttaccagcagtggggtcacatcggtatcgtttacgatggtggtaacaccagcaccttcaccatcctggaacagaactggaacggttacgcgaacaaaaaaccgaccaaacgtgttgataactactacggtctgacccacttcatcgaaatcccggttaaagcgccgccgggtaccgttgctcagtctgctccgaacctggctggttctcgttcttaccgtgaaaccggtaccatgaccgttaccgttgacgctctgaacgttcgtcgtgctccgaacacctctggtgaaatcgttgctgtttacaaacgtggtgaatctttcgactacgacaccgttatcatcgacgttaacggttacgtttgggtttcttacatcggtggttctggtaaacgtaactacgttgctaccggtgctaccaaagacggtaaacgtttcggtaacgcttggggtaccttcaaataa

SEQ ID NO: 43 - DNA, 795 bp, synthetic construct
  source 1..795: mol_type = other DNA
atggcgaaaacccaggcggaaatcaacaaacgtctggatgcgtacgcgaaaggtaccgttgatagcccgtaccgtatcaaaaaagcgacctcttacgatccggctttcggtgttatggaagcgggtgcgatcgatgcggatggttacgctcacgcgcagtgccaggatctgatcaccgattacgttctgtggctgaccgataacaaagttcgtacctggggtaacgcgaaagatcagatcaaacagtcttacggtaccggtttcaaaatccacgaaaacaaaccgtctaccgttccgaaaaaaggttggatcgcggttttcaccagcggttcttaccagcagtggggtcacatcggtatcgtttacgatggtggtaacaccagcaccttcaccatcctggaacagaactggaacggttacgcgaacaaaaaaccgaccaaacgtgttgataactactacggtctgacccacttcatcgaaatcccggttaaagcgccgccgggtaccgttgctcagtctgctccgaacctggctggttctcgttcttaccgtgaaaccggtaccatgaccgttaccgttgacgctctgaacgttcgtcgtgctccgaacacctctggtgaaatcgttgctgtttacaaacgtggtgaatctttcgactacgacaccgttatcatcgacgttaacggttacgtttgggtttcttacatcggtggttctggtaaacgtaactacgttgctaccggtgctaccaaagacggtaaacgtttcggtaacgcttggggtaccttcaaataa

SEQ ID NO: 44 - DNA, 795 bp, synthetic construct
  source 1..795: mol_type = other DNA
atggcgaaaacccaggcggaaatcaacaaacgtctggatgcgtacgcgaaaggtaccgttgatagcccgtaccgtatcaaaaaagcgacctcttacgatccggctttcggtgttatggaagcgggtgcgatcgatgcggatggttactaccacgcgcagtgccaggatctgatcaccgattacgttctgtggctgaccgataacaaagttgctacctggggtaacgcgaaagatcagatcaaacagtcttacggtaccggtttcaaaatccacgaaaacaaaccgtctaccgttccgaaaaaaggttggatcgcggttttcaccagcggttcttaccagcagtggggtcacatcggtatcgtttacgatggtggtaacaccagcaccttcaccatcctggaacagaactggaacggttacgcgaacaaaaaaccgaccaaacgtgttgataactactacggtctgacccacttcatcgaaatcccggttaaagcgccgccgggtaccgttgctcagtctgctccgaacctggctggttctcgttcttaccgtgaaaccggtaccatgaccgttaccgttgacgctctgaacgttcgtcgtgctccgaacacctctggtgaaatcgttgctgtttacaaacgtggtgaatctttcgactacgacaccgttatcatcgacgttaacggttacgtttgggtttcttacatcggtggttctggtaaacgtaactacgttgctaccggtgctaccaaagacggtaaacgtttcggtaacgcttggggtaccttcaaataa

SEQ ID NO: 45 - DNA, 795 bp, synthetic construct
  source 1..795: mol_type = other DNA
atggcgaaaacccaggcggaaatcaacaaacgtctggatgcgtacgcgaaaggtaccgttgatagcccgtaccgtatcaaaaaagcgacctcttacgatccggctttcggtgttatggaagcgggtgcgatcgatgcggatggttactaccacgcgcagtgccaggatctgatcaccgattacgttctgtggctgaccgataacaaagttcgtgcttggggtaacgcgaaagatcagatcaaacagtcttacggtaccggtttcaaaatccacgaaaacaaaccgtctaccgttccgaaaaaaggttggatcgcggttttcaccagcggttcttaccagcagtggggtcacatcggtatcgtttacgatggtggtaacaccagcaccttcaccatcctggaacagaactggaacggttacgcgaacaaaaaaccgaccaaacgtgttgataactactacggtctgacccacttcatcgaaatcccggttaaagcgccgccgggtaccgttgctcagtctgctccgaacctggctggttctcgttcttaccgtgaaaccggtaccatgaccgttaccgttgacgctctgaacgttcgtcgtgctccgaacacctctggtgaaatcgttgctgtttacaaacgtggtgaatctttcgactacgacaccgttatcatcgacgttaacggttacgtttgggtttcttacatcggtggttctggtaaacgtaactacgttgctaccggtgctaccaaagacggtaaacgtttcggtaacgcttggggtaccttcaaataa

SEQ ID NO: 46 - DNA, 795 bp, synthetic construct
  source 1..795: mol_type = other DNA
atggcgaaaacccaggcggaaatcaacaaacgtctggatgcgtacgcgaaaggtaccgttgatagcccgtaccgtatcaaaaaagcgacctcttacgatccgagcttcggtgttatggaagcgggtgcgatcgatgcggatggttacgctcacgcgcagtgccaggatctgatcaccgattacgttctgtggctgaccgataacaaagttgctacctggggtaacgcgaaagatcagatcaaacagtcttacggtaccggtttcaaaatccacgaaaacaaaccgtctaccgttccgaaaaaaggttggatcgcggttttcaccagcggttcttaccagcagtggggtcacatcggtatcgtttacgatggtggtaacaccagcaccttcaccatcctggaacagaactggaacggttacgcgaacaaaaaaccgaccaaacgtgttgataactactacggtctgacccacttcatcgaaatcccggttaaagcgccgccgggtaccgttgctcagtctgctccgaacctggctggttctcgttcttaccgtgaaaccggtaccatgaccgttaccgttgacgctctgaacgttcgtcgtgctccgaacacctctggtgaaatcgttgctgtttacaaacgtggtgaatctttcgactacgacaccgttatcatcgacgttaacggttacgtttgggtttcttacatcggtggttctggtaaacgtaactacgttgctaccggtgctaccaaagacggtaaacgtttcggtaacgcttggggtaccttcaaataa

SEQ ID NO: 47 - DNA, 795 bp, synthetic construct
  source 1..795: mol_type = other DNA
atggcgaaaacccaggcggaaatcaacaaacgtctggatgcgtacgcgaaaggtaccgttgatagcccgtaccgtatcaaaaaagcgacctcttacgatccgagcttcggtgttatggaagcgggtgcgatcgatgcggatggttacgctcacgcgcagtgccaggatctgatcaccgattacgttctgtggctgaccgataacaaagttcgtgcttggggtaacgcgaaagatcagatcaaacagtcttacggtaccggtttcaaaatccacgaaaacaaaccgtctaccgttccgaaaaaaggttggatcgcggttttcaccagcggttcttaccagcagtggggtcacatcggtatcgtttacgatggtggtaacaccagcaccttcaccatcctggaacagaactggaacggttacgcgaacaaaaaaccgaccaaacgtgttgataactactacggtctgacccacttcatcgaaatcccggttaaagcgccgccgggtaccgttgctcagtctgctccgaacctggctggttctcgttcttaccgtgaaaccggtaccatgaccgttaccgttgacgctctgaacgttcgtcgtgctccgaacacctctggtgaaatcgttgctgtttacaaacgtggtgaatctttcgactacgacaccgttatcatcgacgttaacggttacgtttgggtttcttacatcggtggttctggtaaacgtaactacgttgctaccggtgctaccaaagacggtaaacgtttcggtaacgcttggggtaccttcaaataa

SEQ ID NO: 48 - DNA, 795 bp, synthetic construct
  source 1..795: mol_type = other DNA
atggcgaaaacccaggcggaaatcaacaaacgtctggatgcgtacgcgaaaggtaccgttgatagcccgtaccgtatcaaaaaagcgacctcttacgatccgagcttcggtgttatggaagcgggtgcgatcgatgcggatggttactaccacgcgcagtgccaggatctgatcaccgattacgttctgtggctgaccgataacaaagttgctgcttggggtaacgcgaaagatcagatcaaacagtcttacggtaccggtttcaaaatccacgaaaacaaaccgtctaccgttccgaaaaaaggttggatcgcggttttcaccagcggttcttaccagcagtggggtcacatcggtatcgtttacgatggtggtaacaccagcaccttcaccatcctggaacagaactggaacggttacgcgaacaaaaaaccgaccaaacgtgttgataactactacggtctgacccacttcatcgaaatcccggttaaagcgccgccgggtaccgttgctcagtctgctccgaacctggctggttctcgttcttaccgtgaaaccggtaccatgaccgttaccgttgacgctctgaacgttcgtcgtgctccgaacacctctggtgaaatcgttgctgtttacaaacgtggtgaatctttcgactacgacaccgttatcatcgacgttaacggttacgtttgggtttcttacatcggtggttctggtaaacgtaactacgttgctaccggtgctaccaaagacggtaaacgtttcggtaacgcttggggtaccttcaaataa

SEQ ID NO: 49 - protein, 264 aa, synthetic construct
  VARIANT 26: note = X at position 26 may be I or V
  VARIANT 35: note = X at position 35 may be S or A
  VARIANT 45: note = X at position 45 may be D or A
  VARIANT 47: note = X at position 47 may be D or A
  VARIANT 50: note = X at position 50 may be Y or A
  VARIANT 56: note = X at position 56 may be D or A
  VARIANT 71: note = X at position 71 may be R or A
  VARIANT 72: note = X at position 72 may be T or A
  VARIANT 113: note = X at position 113 may be E or Q
  VARIANT 195: note = X at position 195 may be A or G
  VARIANT 199: note = X at position 199 may be R or A
MAKTQAEINKRLDAYAKGTVDSPYRXKKATSYDPXFGVMEAGAIXAXGYXHAQCQXLITDYVLWLTDNKVXXWGNAKDQIKQSYGTGFKIHENKPSTVPKKGWIAVFTSGSYXQWGHIGIVYDGGNTSTFTILEQNWNGYANKKPTKRVDNYYGLTHFIEIPVKAPPGTVAQSAPNLAGSRSYRETGTMTVTVDXLNVXRAPNTSGEIVAVYKRGESFDYDTVIIDVNGYVWVSYIGGSGKRNYVATGATKDGKRFGNAWGTFK